(19) United States
(12) Patent Application Publication
(10) Pub. No.: US 2008/0039904 A1
(43) Pub. Date: Feb. 14, 2008

(54) INTRAVASCULAR IMPLANT SYSTEM

(76) Inventors: Cherik Bulkes, Sussex, WI (US); Stephen Denker, Mequon, WI (US); Arthur J. Beutler, Greendale, WI (US)

Correspondence Address: QUARLES & BRADY LLP, 411 E. WISCONSIN AVENUE, SUITE 2040, MILWAUKEE, WI, 53202-4497, US

(21) Appl. No.: 11/835,075

(22) Filed: Aug. 7, 2007

Related U.S. Application Data
(60) Provisional application No. 60/821,776, filed on Aug. 8, 2006.

(51) Int. Cl. A61N 1/32; A61N 1/05

(52) U.S. Cl. 607/62; 607/116

(57) ABSTRACT

An intravascular implantable system for providing electrical stimulation of tissue inside an animal to deal with a clinical condition is described. The system comprises a power supply module supplying energy to the implantable system, an implanted control module controlling operation of the implantable system and producing desired digital waveforms. Each desired digital waveform has an envelope with a predetermined attribute. An implanted intravascular sensing module sensing at least one parameter of interest for the purpose of dealing with the clinical condition. An intravascular stimulation module is provided to electrically stimulate the tissue with an output waveform that is substantially similar to the desired digital waveform produced by the control module.

### CROSS-REFERENCE TO RELATED APPLICATIONS

This application claims benefit of U.S. Provisional Patent Application No. 60/821,776, filed on Aug. 8, 2006.

### STATEMENT REGARDING FEDERALLY SPONSORED RESEARCH OR DEVELOPMENT

Not Applicable

### BACKGROUND OF THE INVENTION

1. Field of Invention

The invention relates generally to medical care, and particularly to medical care rendered based upon an intravascular implanted device, and more particularly to such care rendered based upon wireless intravascular implants in various body parts, tissues and anatomies. The invention describes an implantable device platform that can be configured for various clinical applications.

2. Description of the Related Art

A wide range of tissues may be monitored and therapeutically treated in a medical field through the use of various types of implants. Over the past decades many such implanted systems have been developed and refined, including cardiac pacemaker systems, which have moved from bulky transcutaneous implants to intravascular implants. Other important uses of implants include implanted cardiac defibrillators, implanted glucose pumps, implanted blood pressure mitigation devices, gastric pacing devices, deep brain stimulators, to mention only a few. In all of these, physiological data is acquired and used for monitoring, alerting and further modulating the therapy. In a typical setting, sensed parameters are also most often presented to a cardiologist or other physician or clinician for use in rendering care.

While such systems provide excellent bases for health care, they have suffered from serious drawbacks, particularly relating to certain types of applications that require minimally invasive procedures to access physiological parameters of interest. Various organs, for example, require surgery to install implants and/or change the battery and replace depleted components. In general, the use of implants is limited to cases where the implants are used primarily for providing therapy. While therapeutic implants have been employed for many years for certain types of data acquisition, such as electrical parameters, certain techniques that have been developed for the implants themselves, such as the data acquisition routines and data analysis protocols, have simply not been conjoined with the use of general physiological parameter determination so as to permit detailed analysis of health conditions.

In an implanted device, there is a need to monitor and/or confirm overall treatment performance and efficacy. In a cardiac pacing device, for example, the monitoring mechanism can determine if pacing is effective and provides rhythm improvement and/or correction. It can monitor physiological signal pattern trends by gathering physiological statistics continuously or periodically against a baseline.

The need for physiological data acquisition for monitoring is especially true in a system with implanted internal components and external components. In such a system, a monitoring mechanism needs to alert the user or a caregiver to invoke a corrective action if the system is compromised in any form, and unable to provide sufficient therapeutic value to the patient. Additionally, it can verify the external device placement issues. The monitoring mechanism can also autonomously initiate communications, in case of emergency, or when preset thresholds for trends or other parameters have been exceeded. The monitoring mechanism can connect to different independent communication targets based on the need. For example, a caretaker can be alerted if internal and external components do not communicate with each other for a predetermined time. As another example, it may contact a medical service or physician if abnormal rhythms are observed. As yet another example, it may trigger a service call if communication is present but battery power is lower than a predetermined value.

There is a significant need in the art for improved procedures and workflows that allow the use of implants in conjunction with certain types of tissues and anatomies so as to permit the use of sophisticated data processing and analysis to render improved health care. Such techniques and improvements would facilitate an entirely new field of health care in the case of organs and tissues that has simply been unavailable either with the use of traditional implants or without.

### Cardiac Applications

### Pacing:

Implantable cardiac devices are well known in the art. They may take the form of implantable defibrillators or cardioverters which treat accelerated rhythms of the heart such as fibrillation or implantable pacemakers which maintain the heart rate above a prescribed limit, such as, for example, to treat a bradycardia. Implantable cardiac devices are also known which incorporate both a pacemaker and a defibrillator.

A pacemaker may be considered as a pacing system. The pacing system is comprised of two major components. One component is a pulse generator which generates the pacing stimulation pulses and includes the electronic circuitry and the power cell or battery. The other component is the lead, or leads, having electrodes which electrically couple the pacemaker to the heart. A lead may provide both unipolar and bipolar pacing polarity electrode configurations. In unipolar pacing, the pacing stimulation pulses are applied between a single electrode carried by the lead, in electrical contact with the desired heart chamber, and the pulse generator case. The electrode serves as the cathode (negative pole) and the case serves as the anode (positive pole). In bipolar pacing, the pacing stimulation pulses are applied between a pair of closely spaced electrodes carried by the lead, in electrical contact with the desired heart chamber, one electrode serving as the anode and the other electrode serving as the cathode.

Pacemakers deliver pacing pulses to the heart to cause the stimulated heart chamber to contract when the patient's own intrinsic rhythm fails. To this end, pacemakers include sensing circuits that sense cardiac activity for the detection of intrinsic cardiac events such as intrinsic atrial events (P waves) and intrinsic ventricular events (R waves). By monitoring such P waves and/or R waves, the pacemaker circuits are able to determine the intrinsic rhythm of the heart and provide stimulation pacing pulses that force atrial and/or ventricular depolarizations at appropriate times in the cardiac cycle when required to help stabilize the electrical rhythm of the heart.

Pacemakers are described as single-chamber or dual-chamber systems. A single chamber system stimulates and senses in one chamber of the heart (atrium or ventricle). A dual chamber system stimulates and/or senses in both chambers of the heart (atrium and ventricle). Dual chamber systems may typically be programmed to operate in either a dual-chamber mode or a single chamber mode.

The energies of the applied pacing pulses must be above the pacing energy stimulation or capture threshold of the respective heart chamber to cause the heart muscle of that chamber to depolarize or contract. If an applied pacing pulse has energy below the capture threshold of the respective chamber, the pacing pulse will be ineffective in causing the heart muscle of the respective chamber to depolarize or contract. As a result, there will be failure in sustaining the pumping action of the heart. It is therefore necessary to utilize applied pacing pulse energies which are assured of being above the capture threshold.

However, it is also desirable to employ pacing energies which are not exorbitantly above the capture threshold. The reason for this is that pacemakers are implanted devices and rely solely on battery power. Using pacing energies that are too much above the capture threshold represent a waste of energy and result in early battery depletion and hence premature device replacement. Capture thresholds are assessed at the periodic follow-up visits with the physician and the output of the pacemaker is adjusted (programmed) to a safety margin that is appropriate based on the results of that evaluation. However, capture thresholds may change between scheduled follow-up visits with the physician. A refinement of the technique of periodic capture threshold measurement by the physician is the automatic performance of capture threshold assessment (automatic capture) and the automatic adjustment of the output of the pulse generator. Capture thresholds may be defined in terms of pulse amplitude, either voltage or current, pulse duration or width, pulse energy, pulse charge or current density. With the introduction of AutoCapture™ by St. Jude Medical Inc., the implanted pacing system periodically and automatically assesses the capture threshold and then adjusts the delivered output. It also monitors capture on a beat-by-beat basis such that a rise in capture threshold will be immediately recognized allowing the system to compensate. Initially, the compensation is in the form of a significantly higher output back-up or safety pulse and then by incrementing the output of the primary pulse until stable capture is again demonstrated. A pacing energy may then be set by adding a small working margin to the capture threshold to assure reliable pacing without rapid depletion of the battery. Without AutoCapture™, a much larger “safety” margin would have to be set and while this may save some energy for the system, it is not as efficient as AutoCapture™ with a small working margin and continued monitoring in minimizing battery current drain and maximizing device longevity.

As is well known in the art, the capture threshold of a heart chamber can, for various reasons, change over time. Hence, pacemakers that incorporate automatic capture are generally able to periodically and automatically perform capture tests. In this way, the variations or changes in capture threshold can be accommodated.

When a pacing pulse is effective in causing depolarization or contraction of the heart muscle, it is referred to as “capture” of the heart. Conversely, when a pacing pulse is ineffective in causing depolarization or contraction of the heart muscle, it is referred to as “lack of capture” or “loss of capture” of the heart.

In one known automatic capture test, the pulse generator applies a succession of primary pacing pulses to the heart at a basic rate. To assess the threshold, the output of the primary pulse is progressively reduced. The output of each successive pair of primary pacing pulses is reduced by a known amount and capture is verified following each pulse. If a primary pulse results in loss of capture, a higher output backup or safety pulse is applied to sustain heart activity. If two consecutive primary pulses at the same output level are associated with loss of capture, the system starts to increment the output associated with the primary pulse. The output of successive primary pacing pulses is then incrementally increased until a primary pacing pulse regains capture. The output of the primary pulse which regains capture is the capture threshold to which the safety margin is added in determining the pacing energy. In these methods, capture may be verified by detecting the evoked response associated with the output pulse, the T-waves, mechanical heart contraction, changes in cardiac blood volume impedance, or another signature of a contracting chamber. Therefore, there is a need for an apparatus that differs significantly from the traditional pacemakers in terms of energy utilization and, therefore, may not require the additional logic for setting capture or automatic capture mechanism. Therefore, the design of pacemaker can be greatly simplified in this regard.

### Defibrillation:

An implantable cardioverter-defibrillator, commonly referred to as an “ICD,” is capable of recognizing tachycardia or fibrillation and delivering electrical therapy to terminate such arrhythmias. ICDs are often configured to perform pacemaking functions as well. A pacemaker generally delivers rhythmic electrical pulses to the heart to maintain a normal rhythm in patients having conduction abnormalities or bradycardia, which is too slow of heart rate. Pathologic tachycardia, which is a rapid heart rate not associated with a normal physiologic response such a response to exercise, is typically treated with low to moderate-energy shocking pulses. The treatment of tachycardia is often referred to as “cardioversion.” Fibrillation is characterized by rapid, unsynchronized depolarizations of the myocardial tissue. Ventricular fibrillation is most often fatal if not treated within a few minutes of its onset. The termination of fibrillation, referred to as “defibrillation,” is accomplished by delivering high-energy shocking pulses.

Upon detection of fibrillation, a defibrillation therapy, referred to herein as a “regimen,” delivered by an implantable defibrillator may include delivery of multiple defibrillation waveforms. Each waveform is defined by a number of parameters including the shape and energy of each pulse. A conventional wave shape is a biphasic waveform in which two pulses that have opposite polarity are generated on the order of 100 microseconds apart. Each waveform within a regimen is delivered on the order of 10 seconds apart. During the time between each defibrillation waveform, the capacitor used for delivering the next waveform is charged, and the defibrillator re-determines if fibrillation is still present. If fibrillation is no longer detected, the regimen is terminated prior to delivering another shock.

Early implantable defibrillation systems required a thoracotomy to allow placement of electrode patches on the epicardial surface of the heart. The risk of morbidity and mortality associated with an open thoracic approach led to the development of transvenous systems that are available today. Transvenous systems include placement of a lead in the right side of the heart with an electrode in the right ventricle, typically near the apex, and a second proximal electrode, typically in the superior vena cava. However, defibrillation using a single lead in the right side of the heart is not successful in all patients and implantation of an epicardial patch is commonly indicated.

The relatively large physical size of early implantable defibrillators, due to large capacitors needed for delivering the high-energy shocks, restricted the implantation of the device to the abdominal region. As capacitor technology has improved, the size of the defibrillators has decreased making pectoral implantation feasible. With the ability to implant the device in the pectoral region, the housing of the device becomes available as an active electrode, sometimes referred to as an “active can,” in combination with the right ventricular lead eliminating the need for an epicardial patch electrode in most patients. Thus, the pectoral implantation of the device overcame the need for a thoracic approach.

Implantable defibrillation systems have been described that use either single or dual defibrillation pathways utilizing combinations of two or three electrodes, selected from a right ventricular lead and the active can. Investigations have been made to determine the optimal defibrillation electrode configuration and results show improved effectiveness of active can configurations, particularly with dual pathway defibrillation using three electrodes.

As the device size continues to be reduced, however, the effectiveness of active can configurations comes into question. Development of coronary sinus electrodes, implanted endovascularly in the area of the left heart, provides additional electrode configurations available for defibrillation. With new configurations available between electrodes implanted in the right ventricle and endovascular electrodes on the left side of the heart, investigation continues for determining the optimal electrode configuration for achieving successful defibrillation at the lowest energy requirement.

However, no single defibrillation electrode configuration will be optimal for all patients. Differences in implant location, patient anatomy and disease state, which can change overtime, will result in different optimal electrode configurations between patients and perhaps within the same patient over time. A given defibrillation pathway selected as the primary pathway based on clinical testing may not continue to be the optimal defibrillation pathway. Therefore, there is a need for apparatus to be adapted to the above mentioned variability without requiring any major surgical procedures.

### Tissue Repair:

Coronary Artery Disease (CAD) affects 1.5 million people in the USA annually. About 10% of these patients die within the first year and about 900,000 suffer from acute myocardial infarction. During CAD, formation of plaques under the endothelial tissue narrows the lumen of the coronary artery and increases its resistance to blood flow, thereby reducing the O2 supply. Injury to the myocardium (i.e., the middle and thickest layer of the heart wall, composed of cardiac muscle) fed by the coronary artery begins to become irreversible within 0.5-1.5 hours and is complete after 6-12 hours, resulting in a condition called acute myocardial infarction (AMI) or simply myocardial infarction (MI).

Myocardial infarction is a condition of irreversible necrosis of heart muscle that results from prolonged ischemia. Damaged or diseased regions of the myocardium are infiltrated with noncontracting scavenger cells and ultimately are replaced with scar tissue. This fibrous scar does not significantly contribute to the contraction of the heart and can, in fact, create electrical abnormalities.

Those who survive AMI have a 4-6 times higher risk of developing heart failure. Current and proposed treatments for those who survive AMI focus on pharmacological approaches and surgical intervention. For example, angioplasty, with and without stents, is a well known technique for reducing stenosis. Most treatments are designed to achieve reperfusion and minimize ventricular damage. However, none of the current or proposed therapies address myocardial necrosis (i.e., degradation and death of the cells of the heart muscle). Because cardiac cells do not divide to repopulate the damaged or diseased region, this region will fill with connective tissue produced by invading fibroblasts. Fibroblasts produce extracellular matrix components of which collagen is the most abundant. Neither the fibroblasts themselves nor the connective tissue they form are contractile. Thus, molecular and cellular cardiomyoplasty research has evolved to directly address myocardial necrosis.

Cellular cardiomyoplasty involves transplanting cells, rather than organs, into the damaged or diseased myocardium with the goal of restoring its contractile function. Molecular cardiomyoplasty has developed because fibroblasts can be genetically manipulated. That is, because fibroblasts, which are not terminally differentiated, arise from the same embryonic cell type as skeletal muscle, their phenotype can be modified, and possibly converted into skeletal muscle satellite cells. This can be done by turning on members of a gene family (myogenic determination genes or “MDGS”) specific for skeletal muscle. A genetically engineered adeno-virus carrying the myogenin gene can be delivered to the MI zone by direct injection. The virus penetrates the cell membrane and uses the cell's own machinery to produce the myogenin protein. Introduction of the myogenin protein into a cell turns on the expression of the myogenin gene, which is a skeletal muscle gene, and which, in turn, switches on the other members of the MDGS and can transform the fibroblast into a skeletal myoblast. To achieve this gene cascade in a fibroblast, replication deficient adenovirus carrying the myogenin gene can be used to deliver the exogenous gene into the host cells. Once the virus infects the fibroblast, the myogenin protein produced from the viral genes turns on the endogenous genes, starting the cascade effect, and converting the fibroblast into a myoblast. Without a nuclear envelope, the virus gets degraded, but the cell's own genes maintain the cell's phenotype as a skeletal muscle cell.

This concept has been well-developed in vitro. However, its viability has not been demonstrated in vivo. Thus, there is a need for an effective system and the method for less invasive delivery of a source of repopulating agents, such as cells or vectors, to the location of the infarct zone of the myocardium and more generally in and/or near damaged or diseased myocardial tissue. Specifically, this involves combining a method of supplying a source of a repopulating agent with a stimulation device. More specifically, this involves the repopulation of the damaged or diseased myocardium with undifferentiated or differentiated contractile cells and augmentation of the newly formed tissue with electrical stimulation to cause the newly formed tissue to contract in synchrony with the heart to improve the cardiac function. Therefore, there is a need for treatment that may be offered to patients post MI via vagal stimulation for regulating rhythm and for healing the tissue potentially through the release of cytokinase.

### Ischemia Detection:

Patients who suffer what is commonly called a heart attack most often experience an episode of myocardial infarction. Myocardial infarction is a necrosis of cardiac tissue brought on by a reduction in blood flow to the infarcted area caused by either an obstruction in an artery or a thrombus in the artery. Early detection of myocardial ischemia provides the opportunity for a wide range of effective therapies such as surgical revascularization, neural stimulation, and drug delivery to reduce cardiac workload or improve cardiac circulation.

Each of the above-mentioned therapeutic techniques is effective in reestablishing blood flow through the effected artery. However, for each therapy, there is a percentage of patients that experience restenosis (reclosure of the artery) after therapy. Restenosis is largely an unpredictable event and the time required for the reclosure to occur may range from a matter of hours to years.

To monitor patients who have suffered from myocardial infarction, physicians may rely upon periodic ECGs (electrocardiograms) which generally require as many as ten leads to be attached to the patient. In addition, after the ECG, physicians then generally require the patient to take a stress test wherein the patient is caused to run on a tread mill until the patient is essentially exhausted to stress the heart. During and after the tread mill exercise the twelve lead is used to determine if the heart continues to receive adequate blood supply while under the stress conditions. Obviously such monitoring is inconvenient to the patient. Physicians may also rely upon Holtor monitoring recordings which may last from 24 to 48 hours. These additional monitoring techniques are equally as inconvenient and in addition, are also annoying. Since all of these monitoring techniques can only be administered periodically at best as a practical matter, and because restenosis and thus future episodes of myocardial infarction are unpredictable events, all too often, a restenosis problem is not detected until the patient experiences pain or suffers an episode of myocardial infarction. Unfortunately, research has shown that pain is not a reliable indicator of ischemia.

There are several methods of myocardial ischemic detection described in literature. One method involves determination of ischemia based on dynamic mechanical heart activity signal and electrical heart activity signal. Another method involves modifying delivery of extra systolic pulse upon detecting ischemia. In another method, a drug delivery system comprises implantable cardiac rhythm management device having ischemia detector, drug level detector, and drug delivery controller. In another system, an implantable cardiac device e.g. pacemaker, for detecting ischemia in patient, has controller storing detection of cardiac ischemia and delivering paces to cardiac chamber based on programmed pacing mode. Yet another myocardial ischemia detecting method, involves detecting cardiac conduction time and determining myocardial ischemia based on detected conduction time measured between the electrodes. Yet another ischemia treatment method involves incrementally altering pacing parameters of cardiac stimulation device by specified amount, on detecting ischemia in patient's heart

Implantable myocardial ischemia detection, indication and action method, in which therapy is initiated, based on data gathered by sensors implanted within subject. Another ischemic condition determination method involves determining ischemic condition based on processed data derived from electric signals of heart during pacing at intrinsic or sensor indicated rate. Another ischemia detection system is integrated with an atrial defibrillator and is responsive to sensed electrical activity of heart for detecting ischemia of heart. Therefore, there is a need for apparatus well suited for the above mentioned applications in a minimally invasive manner.

### CHF—Resynchronization Therapy:

When functioning properly, the human heart maintains its own intrinsic rhythm, and is capable of pumping adequate blood throughout the body's circulatory system. However, some people have abnormal cardiac electrical conduction patterns and irregular cardiac rhythms that are referred to as cardiac arrhythmias. Such arrhythmias result in diminished blood circulation. One mode of treatment includes use of a cardiac rhythm management system. Such systems are often implanted in a patient and deliver electrical stimulation therapy to the patient's heart.

Cardiac rhythm management systems include, among other things, pacemakers, also referred to as pacers. Pacemakers deliver timed sequences of low energy electrical stimuli, called pacing pulses, to the heart, typically via one or more intravascular lead wires or catheters (referred to as “leads”) each having one or more electrodes disposed in or about the heart. Heart contractions are initiated in response to such pacing pulses (this is referred to as “capturing” the heart). Pacemakers also sense electrical activity of the heart in order to detect depolarization signals corresponding to the electrical excitation associated with heart contractions. This function is referred to as cardiac sensing. Cardiac sensing is used to time the delivery of pacing pulses with the heart's intrinsic (native) rhythm. By properly timing the delivery of pacing pulses, the heart can be induced to contract in a proper rhythm, greatly improving its output of blood. Pacemakers are often used to treat patients with bradyarrhythmias (also referred to as bradycardias), that is, hearts that beat too slowly. For that application, the pacemakers may operate in an “on-demand” mode, such that a pacing pulse is delivered to the heart only in absence of a normally timed intrinsic contraction. The on-demand pacing function is often embodied in algorithms exhibiting pace inhibition, in which pacing in a lead is prevented (inhibited) for one heart beat when a cardiac depolarization is detected in the same lead prior to the pace. In bradycardia patients, for example, on-demand pacing can ensure that pacing pulses are delivered only when the patient's intrinsic heart rate drops below a predetermined minimum pacing rate limit, referred to as a lower rate limit (LRL). Some pacemakers provide for two lower rate limits, a first LRL, sometimes called a normal LRL, to provide a minimum necessary heart rate during awake or exercise periods, and a second LRL, sometimes called a hysteresis LRL, to allow the heart to reach naturally slower rates during sleep. When the patent's heart rate falls below the hysteresis LRL, the pacemaker switches to the normal LRL to ensure the patient will have sufficient cardiac output by protecting the patient against abnormally slow heart rates.

Cardiac rhythm management systems also include cardioverters/defibrillators that are capable of delivering higher energy electrical stimuli to the heart. Defibrillators are often used to treat patients with tachyarrhythmias (also referred to as tachycardias), that is, hearts that beat too quickly. Such too-fast heart rhythms also cause diminished blood circulation because the heart is not allowed sufficient time to fill with blood before contracting to expel the blood. Such pumping by the heart is inefficient. A defibrillator is capable of delivering a high energy electrical stimulus that is sometimes referred to as a defibrillation counter shock. The counter shock interrupts the tachyarrhythmia and allows the heart to reestablish a normal rhythm for efficient pumping of blood.

Cardiac rhythm management systems also include, among other things, pacemaker/defibrillators that combine the functions of pacemakers and defibrillators, drug delivery devices, and any other implantable or external systems or devices for diagnosing or treating cardiac arrhythmias.

One problem faced by cardiac rhythm management systems is the treatment of congestive heart failure (also referred to as “CHF”). Congestive heart failure (CHF), or heart failure, is a condition in which the heart can't pump enough blood to the body's other organs. This can result from various causes including narrowed arteries that supply blood to the heart muscle, the coronary artery disease; post heart attack, or myocardial infarction, with scar tissue that interferes with the heart muscle's normal work; high blood pressure; heart valve disease due to past rheumatic fever or other causes; primary disease of the heart muscle itself, called cardiomyopathy; heart defects present at birth—congenital heart defects; and infection of the heart valves and/or heart muscle itself—endocarditis and/or myocarditis.

The “failing” heart keeps working but not as efficiently as it should. People with heart failure can't exert themselves because they become short of breath and tired. By way of example, suppose the muscle in the walls of the left side of the heart deteriorates. As a result, the left atrium and left ventricle become enlarged, and the heart muscle displays less contractility, often associated with unsynchronized contraction patterns. This decreases cardiac output of blood, and in turn, may result in an increased heart rate and less resting time between heart contractions. This condition may be treated by conventional dual chamber pacemakers and a new class of biventricular (or multisite) pacemakers that are termed cardiac resynchronization therapy (CRT) devices. A conventional dual-chamber pacemaker typically paces and senses one atrial chamber and one ventricular chamber. A pacing pulse is timed to be delivered to the ventricular chamber at the end of a programmed atrio-ventricular delay, referred to as AV delay, which is initiated by a pace delivered to or an intrinsic depolarization detected from the atrial chamber. This mode of pacing is sometimes referred to as an atrial tracking mode. The heart can be paced with a shortened AV delay to increase the resting time between heart contractions to increase the amount of blood that fills the ventricular chamber, thus increasing the cardiac output. Biventricular or other multisite CRT devices can pace and sense three or four chambers, usually including the right atrial chamber and both right and left ventricular chambers. By pacing both right and left ventricular chambers, the CRT device can restore a more synchronized contraction of the weakened heart muscle, thus increasing the heart's efficiency as a pump. When treating CHF either with conventional dual-chamber pacemakers or CRT devices, it is critical to pace the ventricular chambers continuously to shorten the AV delay or to provide resynchronizing pacing, otherwise the patient will not receive the intended therapeutic benefit. Thus the intention for treating CHF patients with continuous pacing therapy is different from the intention for treating bradycardia patients with on-demand pacing therapy, which is active only when the heart's intrinsic (native) rhythm is abnormally slow. Therefore, there is a need for conveniently using coronary sinus for deployment of stimulation treatment. Unlike the prior art methods, one may now stimulate left atrium and left and or right ventricle. This treatment has the potential to further improve mitral insufficiency.

Conventional pacemakers and CRT devices in current use rely on conventional on-demand pacing modes to deliver ventricular pacing therapy. These devices need to be adapted to provide a continuous pacing therapy required for treatment of CHF patients. One particular problem in these devices is that they prevent pacing when the heart rate rises above a maximum pacing limit. One such maximum pacing limit is a maximum tracking rate (MTR) limit. “MTR” and “MTR interval,” where an “MTR interval” refers to a time interval between two pacing pulses delivered at the MTR, are used interchangeably, depending on convenience of description, throughout this document. The MTR presents a problem particularly for CHF patients, who typically have elevated heart rates to maintain adequate cardiac output. When a pacemaker or CRT device operates in an atrial tracking mode, it senses the heart's intrinsic rhythm that originates in the right atrial chamber, that is, the intrinsic atrial rate. As long as the intrinsic atrial rate is below the MTR, the device will pace one or both ventricular chambers after an AV delay. If the intrinsic atrial rate rises above the MTR, the device will limit the time interval between adjacent ventricular pacing pulses to an interval corresponding to the MTR, that is, ventricular pacing rate will be limited to the MTR. In this case, the heart's intrinsic contraction rate is faster than the maximum pacing rate allowed by the pacing device so that after a few beats, the heart will begin to excite the ventricles intrinsically at the faster rate, which causes the device to inhibit the ventricular pacing therapy due to the on-demand nature of its pacing algorithm. The MTR is programmable in most conventional devices so that the MTR can be set above the maximum intrinsic atrial rate associated with the patient's maximum exercise level, that is, above the physiological maximum atrial rate. However, many patients suffer from periods of pathologically fast atrial rhythms, called atrial tachyarrhythmia. Also some patients experience pacemaker-mediated tachycardia (PMT), which occurs when ventricular pacing triggers an abnormal retrograde impulse back into the atrial chamber that is sensed by the pacing device and triggers another ventricular pacing pulse, creating a continuous cycle of pacing-induced tachycardia. During these pathological and device-mediated abnormally elevated atrial rhythms, the MTR provides a protection against pacing the patient too fast, which can cause patient discomfort and adverse symptoms. Thus, to protect the patient against abnormally fast pacing, the MTR often is programmed to a low, safe rate that is actually below the physiological maximum heart rate. For many CHF patients with elevated heart rates, this means that they cannot receive the intended pacing therapy during high but physiologically normal heart rates, thus severely limiting the benefit of pacing therapy and the level of exercise they can attain. Therefore, there is a need for addressing this MTR-related problem in therapeutic devices for CHF patients as well as other patients for whom pacing should not be suspended during periods of fast but physiologically normal heart rates. Therefore, there is a need for treating the CHF patients. The heart rate can be slowed down by vagal stimulation.

### CHF—Non-Pharmacologic Inotropic Stimulation:

Prolongation of membrane depolarization by voltage-clamp techniques applied to isolated superfused cardiac muscle have long been known to increase transsarcolemmal calcium entry and thus enhance contractility. Because voltage-clamp techniques are not applicable in situ, this approach has not been explored as a means of enhancing contractility of the intact heart, although, if possible, such an approach might have application as a therapy for heart failure. It has recently been demonstrated that extracellularly applied electric signals have a similar effect as voltage clamping in muscles isolated from normal animals and failing human hearts. When applied regionally, electrical currents can enhance contractility of normal and failing hearts in situ. Preliminary evidence suggests that such cardiac contractility modulating (CCM) signals can also increase contractility in patients with heart failure. Furthermore, locally applied electrical currents are found to enhance global cardiac contractility via regional changes in myocardial contractility without impairing relaxation in situ.

Many device-based therapies are now being investigated for treating the growing number of heart failure patients because despite improved pharmacological therapies, heart failure remains a progressive disorder. Effective therapies that can be deployed relatively non-invasively have the potential for relatively wide-spread application. Preliminary results suggest that this may be effective in improving ventricular contractility and exercise tolerance in heart failure patients having baseline conduction delays with long QRS durations. The technology to deliver CCM therapy can also be implemented in a pacemaker-like device and in principle could be applicable to a significantly larger group of patients because the inotropic effects are not restricted to patients with baseline conduction delays. Specifically, this can be done by having pacing stents in several branches of the coronary sinus to regionally stimulate a heart.

### Vagal Stimulation for Supraventricular Tachycardia Treatment:

Supraventricular tachycardia (SVT) includes abnormally rapid rhythms originating above the ventricles, the lower chambers of the heart. These include atrial fibrillation, AV nodal re-entrant tachycardia, and Wolff-Parkinson-White syndrome. These arrhythmias of atrial chambers can lead to serious performance deficit in the ventricles. Ventricles that receive less than adequate levels of blood begin to contract at ever increasing rates per minute. Ventricles speed up because sensory information processed in the brain indicates that inadequate blood circulation is happening. When heart beat cycles become too fast the heart can go into fibrillation which further cuts the oxygen supply and eventually leads to mortality.

Fibrillation is an exceedingly rapid, but disorganized, contraction or twitching of the heart muscle resulting in grossly inefficient contraction of the myocardium. Especially in the atrial chambers the twitching is vermicular and tends to evolve into rapid circular electrical activation rather than the more normal slower linear conduction. The myocardium quivers during fibrillation and blood circulation falls off severely. The normally coordinated electrical contraction of the myocardium degrades to chaotic electrical conduction which seemly cannot correct itself without critical medicinal and/or electrical intervention.

SVT generally begins and ends quickly. Many people experience short periods of SVT and have no symptoms. However, SVT becomes a problem when it occurs frequently or lasts for long periods of time and produces symptoms. Common symptoms associated with SVT include palpitations, light headedness, and chest pain. SVT may also cause confusion or loss of consciousness.

Treatment of SVT is aimed at correcting the cause of the arrhythmia or controlling the rapid heart rates. SVT can occur because of poor oxygen flow to the heart muscle, lung disease, electrolyte imbalances, high levels of certain medications in the patient, abnormalities of the heart's electrical conduction system, or structural abnormalities of the heart. However, if there is no apparent cause for the SVT, methods of controlling the periods of rapid heart rates are tried. Medications are generally helpful in maintaining a normal heart rhythm. Interventions such as cardioversion or electrophysiology study/catheter ablation may be required to control the SVT.

As an example of SVT, atrial fibrillation (AF) is the most common arrhythmia in humans and represents a significant public health problem. There are presently 2.2 million cases of AF in the United States and approximately 160,000 new cases diagnosed each year. AF is typically managed by a combination of anti-arrhythmic drugs and external or internal electrical cardioversion. In addition, surgical compartmentalization or radio frequency ablation of atrial tissue can be used. Unfortunately, long term success rates are low; AF recurrence is high with both drug treatment and electrical cardioversion with internal and external shocks.

Internal electrical cardioversion of AF remains an uncomfortable therapy option for managing patients with AF. Even with recent advancements, shock voltages necessary to defibrillate the atrial, while considerably lower than that for the ventricles, are still beyond the pain threshold. One reason high voltages may be necessary is that the main generator for AF is the left atrium and direct access to the left atrium is problematic because of the risk of embolism. Typically, atrial defibrillation lead locations are limited to right side chambers (right atrium and right ventricle) and venous structures accessible from the right side of the heart (coronary sinus).

To create a trans-atrial shocking vector, the most common approach is to shock between one or more electrodes on the right side of the heart (right atrial appendage, superior vena cava, or right ventricle) to an electrode on the left side of the heart in the distal coronary sinus. The left atrium is also an important atrial chamber to defibrillate since (i) it can fibrillate independent of the right atrium, (ii) mapping studies have shown that earliest sites of activation following failed defibrillation arise from the left atrium for most defibrillation electrode configurations, (iii) early sites in or near the pulmonary veins have been shown to be responsible for the initiation of and early reoccurrence of AF in many patients, and (iv) ablation of right atrial structures alone has had poor success in terminating AF or preventing its reoccurrence. Nevertheless, there remains a need for means of defibrillating the atria of a subject without unduly high energy defibrillation pulses that would be painful to the subject being treated.

Referring back to the treatment options for SVT, the vagus nerve in the case of supraventricular tachycardia treatment is actually the output of “efferent” nerve. The carotid artery bifraction (where the artery splits the blood supply into two arterial pathways) contains two sensors that we are stimulating. They are the carotid sinus and the carotid body which have sensory nerves that lead to the medulla oblongata with instructions. Afferent nerve is an input informational nerve that provides information to the medulla to help it select the appropriate out put signal that travels, in this case, to the heart.

The vagus nerve contains both afferent and efferent nerves in its bundle. There are some 100,000 fibers in the vagus. About 75% of the fibers are afferent sensors. The balance is the output efferent nerves that travel to all the internal organs that keep the body alive.

Therefore, there is a need to stimulate nerves leading to circuits that would slow down aberrant rhythms in the heart and offer an immediate treatment modality for patients using a less invasive intravascular implant device that provides vagal stimulation. In the case of AV nodal reentry, the vagal stimulation will not just slow the ventricular heart rate, but also terminate the abnormal rhythm. No existing implant uses this technique and it could spare people medications or ablation therapies.

### Brain Applications

### Neurodegenerative Disease Treatment:

Neuroscientists have recognized and continue to explore excitotoxicity, a phenomenon referring to excessive excitation of nerve cells leading to degeneration of the nervous system. This phenomenon has been used to explain cell loss after stroke or some other hypoxic event. The research has focused on nerve cells that have glutamate neurotransmitter receptors especially susceptible to the sustained insult. Hyper excitation of these nerve cells is fundamental to the mechanism. Researchers have also used excitotoxicity to explain the observed cell loss in the CA1 region of the Horn of Ammon in the dentate gyrus of hippocampus in patients and animal subjects that have suffered from seizure activity. Seizures can be viewed as a form of abnormal over excitation of the nerve cells in this region.

Typically, neuroscientists have focused on nerve cells that use the transmitter substance glutamate to communicate with target nerve cells; however, other excitatory amino acids (EAA) are included. When nerve cells are abnormally active, experiencing a lot of action potentials, they are believed to release excessive amounts of glutamate or other EAA at their synaptic terminals. The presence of excessive amounts of glutamate leads to toxic effects on the secondary nerve cells targeted by the hyperactive ones. These toxic effects are believed to be mediated by an accumulation of calcium.

It has shown that stimulation of the Vim nucleus of the Thalamus will block tremor. In this instance, stimulation at frequencies around 100 to 185 pulses per second accomplishes the same physiological response as a lesion of this region. Thus, it appears that stimulation inhibits the output of these cells. Similarly stimulation of the subthalamus can be performed.

Parkinson's disease is the result of degeneration of the substantia nigra pars compacta. The cells of subthalamus have been shown to use glutamate as the neurotransmitter effecting communication with their target cells of the basal ganglia. The state of hyperexcitation that exists in Parkinson's disease will cause an excessive release of glutamate. This, in theory, will lead to further degeneration via the mechanism described above.

A method of arresting degeneration of the substantia nigra involves high frequency electrical pulsing of the subthalamic nucleus to block stimulation of the subthalamic nucleus, thereby inhibiting excessive release of glutamate at the terminal ends of the axons projecting from the subthalamic nucleus to the substantia nigra. Therefore, there is a need to treat a neurodegenerative disorder, such as Parkinson's disease, by means of an apparatus by therapeutically stimulating the brain.

### Epilepsy Alerting

Epilepsy, a neurological disorder characterized by the occurrence of seizures (specifically episodic impairment or loss of consciousness, abnormal motor phenomena, psychic or sensory disturbances, or the perturbation of the autonomic nervous system), is debilitating to a great number of people. It is believed that as many as two to four million Americans may suffer from various forms of epilepsy. Research has found that its prevalence may be even greater worldwide, particularly in less economically developed nations, suggesting that the worldwide figure for epilepsy sufferers may be in excess of one hundred million.

Because epilepsy is characterized by seizures, its sufferers are frequently limited in the kinds of activities they may participate in. Epilepsy can prevent people from driving, working, or otherwise participating in much of what society has to offer. Some epilepsy sufferers have serious seizures so frequently that they are effectively incapacitated.

Furthermore, epilepsy is often progressive and can be associated with degenerative disorders and conditions. Over time, epileptic seizures often become more frequent and more serious, and in particularly severe cases, are likely to lead to deterioration of other brain functions (including cognitive function) as well as physical impairments.

The current state of the art in treating neurological disorders, particularly epilepsy, typically involves drug therapy and surgery. The first approach is usually drug therapy.

A number of drugs are approved and available for treating epilepsy, such as sodium valproate, phenobarbital/primidone, ethosuximide, gabapentin, phenytoin, and carbamazepine, as well as a number of others. Unfortunately, those drugs typically have serious side effects, especially toxicity, and it is extremely important in most cases to maintain a precise therapeutic serum level to avoid breakthrough seizures (if the dosage is too low) or toxic effects (if the dosage is too high). The need for patient discipline is high, especially when a patient's drug regimen causes unpleasant side effects the patient may wish to avoid.

Moreover, while many patients respond well to drug therapy alone, a significant number (at least 20-30%) do not. For those patients, surgery is presently the best-established and most viable alternative course of treatment.

Currently practiced surgical approaches include radical surgical resection such as hemispherectomy, corticectomy, lobectomy and partial lobectomy, and less-radical lesionectomy, transection, and stereotactic ablation. Besides being less than fully successful, these surgical approaches generally have a high risk of complications, and can often result in damage to eloquent (i.e., functionally important) brain regions and the consequent long-term impairment of various cognitive and other neurological functions. Furthermore, for a variety of reasons, such surgical treatments are contraindicated in a substantial number of patients. And unfortunately, even after radical brain surgery, many epilepsy patients are still not seizure-free.

Electrical stimulation is an emerging therapy for treating epilepsy. However, currently approved and available electrical stimulation devices apply continuous electrical stimulation to neural tissue surrounding or near implanted electrodes, and do not perform any detection—they are not responsive to relevant neurological conditions.

The NeuroCybernetic Prosthesis (NCP) from Cyberonics, for example, applies continuous electrical stimulation to the patient's vagus nerve. This approach has been found to reduce seizures by about 50% in about 50% of patients. Unfortunately, a much greater reduction in the incidence of seizures is needed to provide clinical benefit. The Activa® device from Medtronic, Inc. of Minneapolis, Minn., USA is a pectorally implanted continuous deep brain stimulator intended primarily to treat Parkinson's disease. In operation, it supplies a continuous electrical pulse stream to a selected deep brain structure where an electrode has been implanted.

A typical epilepsy patient experiences episodic attacks or seizures, which are generally defined as periods of abnormal neurological activity. As is traditional in the art, such periods shall be referred to herein as “ictal” (though it should be noted that “ictal” can refer to neurological phenomena other than epileptic seizures).

Known work on detection and treatment of epilepsy via electrical stimulation has focused on a region of the brain frequently referred to as an epileptic (or epileptogenic) focus, particularly in patients suffering from partial epilepsy (the most common form of adult-onset epilepsy). In at least some partial epilepsy sufferers, it is the area where hyper synchronous activity consistently begins; it typically spreads outward, and into other regions of the brain, from there. The characteristics of an epileptic seizure onset are different from patient to patient, but are frequently consistent from seizure to seizure within a single patient. Although seizures in a partial epilepsy sufferer frequently begin in the same region of the brain, they may secondarily generalize quickly to cover a significant portion of the brain. Patients with primary generalized epilepsy may not have any specific identifiable seizure origin.

Unfortunately, continuous stimulation of deep brain structures for the treatment of epilepsy has not met with consistent success. To be effective in terminating seizures, it has traditionally been believed that epilepsy stimulation should be performed near the focus of the epileptogenic region. The focus is often in the neocortex, where continuous stimulation may cause significant neurological deficit with clinical symptoms including loss of speech, sensory disorders, or involuntary motion. Accordingly, research has been directed toward automatic responsive epilepsy treatment at or near the focus, based on a detection of imminent seizure.

Recent research, however, indicates that the concept of a single epileptic focus does not necessarily accurately reflect the origins of partial epilepsy, at least in humans. The human brain is a complex system, and although an anomalous signal may first be detected via known methods at a particular location or region, that does not necessarily imply that area is the true epileptogenic origin of an epileptic seizure. Nor is the region where abnormal signals are first identified necessarily the location where it is most effective to treat a seizure or its precursor. In fact, it is possible to have multiple locations in a single patient's brain that all act as epileptic foci. And in generalized seizures, abnormal EEG signals can be found throughout a patient's brain practically simultaneously.

Most prior work on the detection and responsive treatment of seizures via electrical stimulation has focused on analysis of electroencephalogram (EEG) and electrocorticogram (ECoG) waveforms. In general, EEG signals represent aggregate neuronal activity potentials detectable via electrodes applied to a patient's scalp, and ECoGs use internal electrodes near the surface of the brain. ECoG signals, deep-brain counterparts to EEG signals, are also detectable via electrodes implanted under the dura mater, and usually within the patient's brain. Unless the context clearly and expressly indicates otherwise, the term “EEG” shall be used generically herein to refer to both EEG and ECoG signals.

Much of the work on detection has focused on the use of time-domain analysis of EEG signals. In a typical time-domain detection system, EEG signals are received by one or more implanted electrodes and then processed by a control module, which then is capable of performing an action (intervention, warning, recording, etc.) when an abnormal event is detected.

In the Gotman system, EEG waveforms are filtered and decomposed into “features” representing characteristics of interest in the waveforms. One such feature is characterized by the regular occurrence (i.e., density) of half-waves exceeding a threshold amplitude occurring in a specified frequency band between approximately 3 Hz and 20 Hz, especially in comparison to background (non-ictal) activity. When such half-waves are detected, the onset of a seizure is identified. A more computationally demanding approach is to transform EEG signals into the frequency domain for rigorous spectrum analysis. Although this approach is generally believed to achieve good results, for the most part, its computational expense renders it less than optimal for use in long-term implanted epilepsy monitor and treatment devices. With current technology, the battery life in an implantable device computationally capable of performing the Dorfmeister method would be too short for it to be feasible.

An alternative and more complex approach analyzes various non-linear and statistical characteristics of EEG signals to identify the onset of ictal activity. Once more, the calculation of statistically relevant characteristics is not believed to be feasible in an implantable device.

Another previous system developed by Robert Fischell describes an implantable seizure detection and treatment system wherein various detection methods are possible, all of which essentially rely upon the analysis (either in the time domain or the frequency domain) of processed EEG signals. In this device a controller is preferably implanted intracranially, but other approaches are also possible, including the use of an external controller. When a seizure is detected, the Fischell system applies responsive electrical stimulation to terminate the seizure, a capability that will be discussed in further detail below.

All of these approaches provide useful information, and in some cases may provide sufficient information for accurate detection and prediction of most imminent epileptic seizures.

Accordingly, as has been previously suggested, it is possible to treat and terminate seizures by applying electrical stimulation to the brain. It should be noted, however, that the epilepsy detection methods described above rely, at least in part, on the continuous analysis of EEG signals. To the extent responsive electrical stimulation is applied in response to a detection of epileptiform activity, artifacts of the stimulation received by the epileptiform activity detector may be significantly disruptive of the detection algorithms. A potential solution to this problem is to blank the sensing amplifiers used to receive EEG signals during and for a period after the application of electrical stimulation, but this will lead to a loss of data during the blanking period.

To recapitulate somewhat, in general, partial epilepsy is a much more complex phenomenon than traditionally thought. It is believed to be advantageous to provide therapeutic electrical stimulation in a number of brain regions involved in a patient's epilepsy, but known approaches do not do this in any meaningful way. Given the neural organization of the brain, in a given patient it may be more effective to stimulate pathways associated with epileptogenic focus, rather than the focus itself, to disrupt or block the epileptiform activity to prevent the occurrence of a clinical seizure. It is anticipated that stimulation from contralateral structures, particularly when the focus is hippocampus, may be the preferred method of treating some types of spontaneously occurring epileptiform activity. In addition, it may be particularly advantageous to apply electrical stimulation exclusively in areas distant from an epileptogenic region, as electrical stimulation of neural tissue that is especially sensitive may contribute to or initiate the hyper synchronous activity that characterizes an epileptic seizure. And furthermore, remote stimulation would serve to advantageously reduce the effects of artifacts on, the epilepsy detection methods employed. Therefore, there is a need for apparatus that may be well suited for this purpose.

### Eating Disorders and Obesity Treatment

Obesity affects millions of Americans, and a substantial percentage of these people are morbidly obese, suffering such obesity-related problems as heart disease, vascular disease, and social isolation. An additional number of Americans suffer from various other eating disorders that may result in cachexia (i.e., a general physical wasting and malnutrition) or periods of obesity and/or cachexia. The etiology of obesity is largely unknown. The etiology of some eating disorders is psychological in many patients, but for other patients, is poorly understood.

Patients suffering from morbid obesity and/or other eating disorders have very limited treatment options. For instance, some of these patients may undergo surgery to reduce the effective size of the stomach (“stomach stapling”) and to reduce the length of the nutrient-absorbing small intestine. Such highly invasive surgery is associated with both acute and chronic complications, including infection, digestive problems, and deficiency in essential nutrients. In extreme cases, patients may require surgical intervention to a put a feeding tube in place. Patients suffering from eating disorders may suffer long-term complications such as osteoporosis. Additional treatment options are needed.

The invention disclosed herein provides systems and methods for introducing one or more stimulating drugs and/or applying electrical stimulation to one or more areas of the brain for treating or preventing obesity and/or other eating disorders, as well as the symptoms and pathological consequences thereof. Proper stimulation of specific sites in the brain via deep brain stimulation may lead to changes in levels or responses to neurotransmitters, hormones, and/or other substances in the body that treat eating disorders. Therefore, there is a need for providing electrical stimulation and sensing for treating these disorders.

### Schizophrenia Treatment:

Within the field of neurosurgery, in many instances, the preferred effect is to stimulate or reversibly block nervous tissue. Electrical stimulation permits such stimulation of the target neural structures, and equally importantly, it does not require the destruction of the nervous tissue (it is a reversible process, which can literally be shut off or removed at will).

Within this field, however, disorders manifesting gross physical dysfunction, not otherwise determinable as having emotional or psychiatric origins, comprise the vast majority of those pathologies treated by deep brain stimulation. A noteworthy example of treatment of a gross physical disorder by electrical stimulation involves reducing, and in some cases eliminating, the tremor associated with Parkinson's disease by the application of a high frequency electrical pulse directly to the subthalamic nucleus.

Conversely, direct neuro-augmentation treatments for disorders, which have traditionally been treated by behavioral therapy or psychiatric drugs, has been largely limited to peripheral nerve stimulation. A noteworthy example is the effort to control compulsive eating disorders by stimulation of the vagus nerve which has been described by Wernicke, et al. in U.S. Pat. No. 5,263,480. This treatment seeks to induce a satiety effect by stimulating the afferent vagal fibers of the stomach. For patients having weak emotional and/or psychological components to their eating disorders, this treatment can be effective insofar as it eliminates the additional (quasi-normal) physio-chemical stimulus to continue eating. This is especially true for patients who exhibit subnormal independent functioning of these fibers of the vagus nerve. For compulsive eating patients who are not suffering from an insufficient level of afferent vagal nerve activity resulting from sufficient food intake, however, the over stimulation of the vagus nerve and potential resultant over abundance of satiety mediating chemicals (cholecystokinin and pancreatic glucagon) may have little effect. It has even been suggested that continued compulsive eating, despite overstimulation of the vagus nerve, may exacerbate the emotional component of the patient's disorder.

The stimulation of a peripheral nerve can result in the release of a chemical which specifically counteracts the psychological pathology, for example if the release of greater amounts of cholecystokinin and pancreatic glucagon had a direct effect on the pathology exhibited in the brain, then, for that patient, the treatment will have a greater probability of success. If, however, as is most probably the case, the increase in the level of activity of the peripheral nerve does not result in the release of such a chemical, and therefore, has no effect on the area of the brain responsible for the emotional/psychiatric component of the disorder, then the treatment will have a much lower probability of success.

The impetus would, therefore, be to treat psychological disorders with direct modulation of activity in that portion of the brain which is causing the pathological behavior. Unfortunately, the ability to determine what region of the brain is responsible for a given patient's disorder is very difficult, and even more importantly, does not usually provide consistent patterns across a population of similarly afflicted patients. By this it is meant that the region of the brain which causes the behavioral pathology of one compulsive eating patient, for example, does not necessarily correspond in any way with the region of another compulsive eating patient.

In some manner, however, the determination of what regions of the brain are exhibiting pathological function must be determined. Fortunately, a method for determining precisely this has been developed by a number of researchers. Normal brain function can be characterized by four discrete frequencies of electrical output. Other frequencies are almost exclusively associated with pathology. The use of magnetoencephalography (MEG scans) has permitted quantification of electrical activity in specific regions of the brain. It has been proposed that MEG scans may be used to identify regions exhibiting pathological electrical activity. The resolution of the MEG scans of the brain are highly accurate (sub-one millimeter accuracy), however, correlating the MEG scan with MRI images for the surgical purposes of identifying anatomical structures limits the overall resolution for surgical purposes to a volume of 10 to 30 cubic millimeters. As stated above, however, simply identifying the regions of the brain which are exhibiting pathological electrical activity for a specific patient is not sufficient to generalize across a large population of patients, even if they are exhibiting identical disorders.

Fortunately, the architecture of the brain provides a substantial advantage in the search for a generic solution. This design advantage takes the form of a centralized signaling nexus through which many of the brain's disparate functions are channeled in an organized and predictable manner. More particularly, the thalamus is comprised of a large plurality (as many as one hundred or more) of nerve bundles, or nuclei, which receives and channels nerve activity from all areas of the nervous system and interconnects various activities within the brain. It is this key which permits the treatment of common psychological disorders by brain stimulation of one specific area, rather than having to customize the (gross) placement of the stimulator for each patient. Therefore, there is a need to provide effective therapy for this disorder.

### Pain Treatment:

The first reports of direct electrical stimulation of the somatosensory thalamus (ventroposterior lateral and medial; VPL/VPM) to treat chronic pain in the human appeared in the early 1970s. This procedure is most often employed for treating neuropathic pain and was used with varying success during the last 3 decades. Nevertheless, despite numerous clinical studies reporting pain relief, the success of thalamic stimulation for the treatment of chronic pain remains unpredictable. Furthermore, evaluation of stimulation-produced pain relief is difficult because there can be a large placebo effect. The ventroposterior thalamus was stimulated in patients suffering from deafferentation pain, based on the theory that such pain is caused by lack of proprioceptive stimuli reaching the thalamus. Stimulating the primary somatosensory pathway at this thalamic site was an effort to compensate for the lack of normal sensory input. The gate control theory further championed the idea that stimulation of low threshold somatosensory pathways inhibits pain; thus direct stimulation of this pathway at the thalamic level would be expected to reduce neuropathic pain, which is characterized by loss of such input after damage in the peripheral or CNS. Physiological studies in anesthetized animals confirmed that stimulation in VPL thalamus inhibits the activity of both spinothalamic nociceptive neurons in monkey and thalamic parafascicular nociceptive neurons in rat. Therefore, there is a need to provide effective therapy for this disorder.

### Neurological Disorder Treatment:

There are a wide range of neurological and psychological disorders for which treatment may be provided by various means. For many disorders, administration of pharmaceutical agents is the most common treatment modality. In cases in which the symptoms of the disorder are resistant to pharmacological treatment or for which no pharmacological treatment exists, other modalities may be used, including neurostimulation.

Neurostimulation is a method of disease treatment which uses an electrical stimulator to provide a current signal which is used to stimulate the central nervous system (CNS), generally either directly or by means of a nerve of the peripheral nervous system. Such neurostimulators and their corresponding electrodes are generally implanted in a patient's body. There are currently two primary methods of neurostimulation for central nervous system disorders; deep brain stimulation (DBS) and vagus nerve stimulation (VNS). Deep brain stimulation uses an electrode implanted directly in a patient's brain, while VNS stimulates a patient's vagus nerve peripherally.

A commercially available neurostimulator is manufactured and sold by Medtronic Inc. as DBS™ model 3386, having a stimulating lead with four cylindrical stimulating electrodes. The deep brain stimulator is a surgically implanted medical device, similar to a cardiac pacemaker, which delivers high-frequency, pulsatile electrical stimulation to precisely targeted areas within the brain. The device consists of a very small electrode array (electrodes 1.5 mm in length with 3 mm center to center separation) placed in a deep brain structure and connected through an extension wire to an electrical pulse generator surgically implanted under the skin near the collarbone. The Medtronic DBS™ has received marketing clearance from the United States Food and Drug Administration (FDA) with an indication for treatment of Parkinson's disease, essential tremor, and dystonia. Current research is evaluating deep brain stimulation as a treatment for epilepsy, psychiatric disorders, and chronic pain.

The deep brain stimulator is surgically placed under the skin of the chest of the patient. The device's stimulating electrode lead is connected to the stimulator wires and is placed in a specific inter-cranial location which may vary depending on the region of the brain being treated. The deep brain stimulator is adjusted by several parameters: (1) location of the 4 electrode lead, (2) selection of the stimulating electrodes, (3) amplitude of the stimulator signal, (4) frequency (repetition rate) of the stimulator signal, (5) polarity of the stimulating signal, and (6) pulse width of the stimulating signal. Post-implantation, all of these parameters except electrode location can be non-invasively varied by a clinician to enhance therapeutic effectiveness and minimize side effects. Amplitude, measured in volts, is the intensity or strength of the stimulation. The typical range is 1.5 to 9.0 volts. Frequency is the repetition rate at which the stimulation pulse is delivered and is measured in pulses per second (Hz); it typically ranges from 100-185 Hz. The pulse width is the duration of the stimulation pulse, measured in microseconds. The average pulse width ranges from 60-120 microseconds.

Another commercially available neurostimulator is designed for use on the peripheral nervous system, specifically the vagus nerve. An example of this type of system is designed and sold by Cyberonics, Inc. in Houston, Tex. U.S.A. The Vagus Nerve Stimulator (VNS) Therapy device is implanted in a patient's chest under the skin immediately below the collarbone or close to the armpit. Two tiny wires from the device wrap around the vagus nerve on the left side of the neck. Through stimulation of this peripheral nerve, brain function is affected. VNS therapy has been granted marketing clearance by the FDA with an indication for treatment of epilepsy and is being investigated to treat a number of other central nervous system diseases and conditions, such as obesity, depression, Alzheimer's disease, etc. Therefore, there is a need to provide effective therapy for this disorder.

### Huntington's Disease Treatment:

Huntington's disease (HD) is an inherited disorder characterized by abnormalities in motor function, personality, thinking, and memory. While the typical age of onset is approximately 40-45, onset may be much earlier. HD is a progressive disorder that leads to death approximately 17 years after onset.

HD is dominantly inherited. The child of a person with HD has a 50% risk of inheriting the gene and thus developing the disorder. The abnormal gene causing HD was discovered in 1993. (HD is specifically caused by an unstable amplification of a trinucleotide [CAG]n repeat with the coding region of the gene.) The gene controls manufacture of a protein that appears to be essential to normal brain function.

The genetic mutation that produces HD causes neurons in parts of the brain to degenerate, causing uncontrollable movements, mental deterioration, and emotional imbalances. Most affected are neurons in the basal ganglia, deep structures within the brain (i.e., caudate nucleus, putamen, globus pallidus, subthalamic nucleus, and substantia nigra) that, among other functions, help coordinate movement. Other degeneration occurs in the cortex, which may affect thought, perception and memory. The discovery of the HD gene is likely to lead to the development of gene based therapeutic strategies; however, gene therapy is still investigational and is likely to remain so for at least another decade. A test to identify carriers of the HD gene is available.

HD has an estimated frequency of 4-7 per 100,000 persons. Up to 30,000 are afflicted in the United States alone. Another 150,000 persons have a 50% percent chance of developing it, and thousands more related to them live within its shadow, knowing of its presence in their family history.

Early symptoms of HD are subtle, can vary from person to person, and are easily overlooked or misinterpreted. The afflicted person may experiences mood swings, become irritable, apathetic, lethargic, depressed or angry. Sometimes these symptoms disappear as the disease progresses; sometimes they develop into hostile outbursts or deep depression. Over time, the patient's judgment, memory, and other cognitive functions begin to deteriorate. He or she may begin to have difficulty driving, keeping track of things, making decisions, or even answering questions. The more the disease progresses, the more the ability to concentrate becomes affected. Uncontrolled movements may develop in the fingers, feet, face, or trunk. These tics are the beginnings of chorea (nervous disorder marked by spasmodic movements of limbs and facial muscles and by incoordination), and can become more intense if the patient is anxious or disturbed.

The classic signs of HD are progressive chorea, rigidity, and dementia, frequently associated with seizures. A characteristic atrophy of the caudate nucleus of the brain is seen radiographically. Typically, there is a prodromal phase of mild psychotic and behavioral symptoms which precedes frank chorea by up to 10 years. However, findings by Shiwach, et al. in 1994 clashed with the conventional wisdom that psychiatric symptoms are a frequent presentation of HD before the development of neurologic symptoms. (see Shiwach, et al. “A Controlled Psychiatric Study Of Individuals At Risk For Huntington's Disease,” British Journal of Psychiatry, 165:500-505, 1994). They performed a control study of 93 neurologically healthy individuals at risk for HD, i.e., who had a parent who developed HD, which means that the child had a 50% chance of developing HD. Genetic test results were available for only 53 of the 93 individuals. The 20 asymptomatic individuals carrying the HD gene (and thus likely to develop HD) showed no increased incidence of psychiatric disease of any sort when compared to the 33 individuals not carrying the HD gene. However, the whole group of normal at-risk individuals showed a significantly greater number of psychiatric episodes than did their 43 spouses, suggesting stress from the uncertainty associated with belonging to a family segregating this disorder. The authors concluded that neither depression nor psychiatric disorders are likely to be significant pre-neurologic indicators of expression of the disease gene.

As the disease progresses, new symptoms begin to emerge: mild clumsiness, loss of coordination, and balance problems. Walking becomes increasingly difficult, and the person may stumble or fall. Speech may become slurred. The patient may begin having trouble swallowing or eating. Gradually, he or she may lose the ability to recognize others, although many HD patients retain an awareness of their surroundings and can express emotions. The illness typically runs its full terminal course in 10 to 30 years. Death often results from pneumonia when the end-stage patient is bedridden. Other patients die from infections or other physical complications including injuries sustained in falls and other accidents.

As mentioned above, a test to identify carriers of the HD gene is available. Imaging studies (e.g., positron emission tomography (PET)) may be used to reveal degeneration of the caudate nucleus of the brain, which is characteristic of HD.

The ultimate goal of Huntington's disease treatment is to prevent the cell death that leads to its devastating symptoms. However, there is no proven way to do this at this point; some medications and gene therapy agents are under investigation. There is currently no cure for Huntington's disease.

Treatment generally focuses on addressing the disease's symptoms, preventing associated complications and providing support and assistance to the patient and those close to him or her. For those diagnosed with HD, physicians often prescribe various medications to help control emotional and movement problems. Clonazepam (and other benzodiazepines) may alleviate choreic movements, and antipsychotic drugs such as haloperidol may help control hallucinations, delusions, or violent outbursts. Antipsychotic drugs are contraindicated if the patient has dystonia, a form of muscular contraction sometimes associated with HD, as it can worsen the condition, causing stiffness and rigidity.

If the patient suffers from depression, the physician may prescribe fluoxetine, sertraline hydrochloride, or nortriptyline. Tranquilizers can be used to treat anxiety, and lithium may be prescribed for patients who exhibit pathological excitement or severe mood swings. Other medications may be prescribed for severe obsessive-compulsive behaviors some individuals with HD develop. Because most drugs used to treat symptoms of HD can produce undesirable side effects, ranging from fatigue to restlessness and hyperexcitability, physicians try to prescribe the lowest possible dose.

In HD, the primary pathological changes are found in the striatum (i.e., the caudate, putamen, and nucleus accumbens), where GABAergic neurons undergo degenerative changes. Clinical trials of fetal striatal tissue transplantation for the treatment of HD are ongoing, but it is yet unproven.

While deep brain stimulation (DBS) has been applied to the treatment of other movement disorders, e.g., Parkinson's disease, deep brain stimulation has yet to be applied to the treatment of Huntington's disease. Relatively few interventions have been pursued in hyperkinetic disorders such as Huntington's disease, mainly owing to the lack of an adequate target nucleus.

With such limited treatment options for Huntington's disease, the inventors believe that additional and improved treatments, with enhanced systems and modified methods, are needed.

### Spine Stimulation:

Chronic pain is usually a multidimensional phenomenon involving complex physiological and emotional interactions. For instance, one type of chronic pain, complex regional pain syndrome (CRPS)—which includes the disorder formerly referred to as reflex sympathetic dystrophy (RSD)—most often occurs after an injury, such as a bone fracture. The pain is considered “complex regional” since it is located in one region of the body (such as an arm or leg), yet can spread to additional areas. Since CRPS typically affects the sympathetic nervous system, which in turn affects all tissue levels (skin, bone, etc.), many symptoms may occur. Pain is the main symptom. Other symptoms vary, but can include loss of function, temperature changes, swelling, sensitivity to touch, and skin changes.

Another type of chronic pain, failed back surgery syndrome (FBSS), refers to patients who have undergone one or more surgical procedures and continue to experience pain. Included in this condition are recurring disc herniation, epidural scarring, and injured nerve roots.

Arachnoiditis, a disease that occurs when the membrane in direct contact with the spinal fluid becomes inflamed, causes chronic pain by pressing on the nerves. It is unclear what causes this condition.

Yet another cause of chronic pain is inflammation and degeneration of peripheral nerves, called neuropathy. This condition is a common complication of diabetes, affecting 60%-70% of diabetics. Pain in the lower limbs is a common symptom.

An estimated 10% of gynecological visits involve a complaint of chronic pelvic pain. In approximately one-third of patients with chronic pelvic pain, no identifiable cause is ever found, even with procedures as invasive as exploratory laparotomy. Such patients are treated symptomatically for their pain.

A multitude of other diseases and conditions cause chronic pain, including postherpetic neuralgia and fibromyalgia syndrome. Neurostimulation of spinal nerves, nerve roots, and the spinal cord has been demonstrated to provide symptomatic treatment in patients with intractable chronic pain.

Many other examples of chronic pain exist, as chronic pain may occur in any area of the body. For many sufferers, no cause is ever found. Thus, many types of chronic pain are treated symptomatically. For instance, many people suffer from chronic headaches/migraine and/or facial pain. As with other types of chronic pain, if the underlying cause is found, the cause may or may not be treatable. Alternatively, treatment may be only to relieve the pain.

All of the devices currently available for producing therapeutic stimulation have drawbacks. Many are large devices that must apply stimulation transcutaneously. For instance, transcutaneous electrical nerve stimulation (TENS) is used to modulate the stimulus transmissions by which pain is felt by applying low-voltage electrical stimulation to large peripheral nerve fibers via electrodes placed on the skin. TENS devices can produce significant discomfort and can only be used intermittently.

Other devices require that a needle electrode(s) be inserted through the skin during stimulation sessions. These devices may only be used acutely, and may cause significant discomfort.

Implantable, chronic stimulation devices are available, but these currently require a significant surgical procedure for implantation. Surgically implanted stimulators, such as spinal cord stimulators, have been described in the art. These spinal cord stimulators have different forms, but are usually comprised of an implantable control module to which is connected a series of leads that must be routed to nerve bundles in the spinal cord, to nerve roots and/or spinal nerves emanating from the spinal cord, or to peripheral nerves. The implantable devices are relatively large and expensive. In addition, they require significant surgical procedures for placement of electrodes, leads, and processing units. These devices may also require an external apparatus that needs to be strapped or otherwise affixed to the skin. Drawbacks, such as size (of internal and/or external components), discomfort, inconvenience, complex surgical procedures, and/or only acute or intermittent use has generally confined their use to patients with severe symptoms and the capacity to finance the surgery.

There are a number of theories regarding how stimulation therapies such as TENS machines and spinal cord stimulators may inhibit or relieve pain. The most common theory—gate theory or gate control theory—suggests that stimulation of fast conducting nerves that travel to the spinal cord produces signals that “beat” slower pain-carrying nerve signals and, therefore, override/prevent the message of pain from reaching the spinal cord. Thus, the stimulation closes the “gate” of entry to the spinal cord. It is believed that small diameter nerve fibers carry the relatively slower-traveling pain signals, while large diameter fibers carry signals of e.g., touch that travel more quickly to the brain.

Spinal cord stimulation (also called dorsal column stimulation) is best suited for back and lower extremity pain related to adhesive arachnoiditis, FBSS, causalgia, phantom limb and stump pain, and ischemic pain. Spinal cord stimulation is thought to relieve pain through the gate control theory described above. Thus, applying a direct physical or electrical stimulus to the larger diameter nerve fibers of the spinal cord should, in effect, block pain signals from traveling to the patient's brain.

The gate control theory has always been controversial, as there are certain conditions such as hyperalgesia, which it does not fully explain. The relief of pain by electrical stimulation of a peripheral nerve, or even of the spinal cord, may be due to a frequency-related conduction block which acts on primary afferent branch points where dorsal column fibers and dorsal horn collaterals diverge. Spinal cord stimulation patients tend to show a preference for a minimum pulse repetition rate of 25 Hz.

Stimulation may also involve direct inhibition of an abnormally firing or damaged nerve. A damaged nerve may be sensitive to slight mechanical stimuli (motion) and/or noradrenaline (a chemical utilized by the sympathetic nervous system), which in turn results in abnormal firing of the nerve's pain fibers. It is theorized that stimulation relieves this pain by directly inhibiting the electrical firing occurring at the damaged nerve ends.

Stimulation is also thought to control pain by triggering the release of endorphins. Endorphins are considered to be the body's own pain-killing chemicals. By binding to opioid receptors in the brain, endorphins have a potent analgesic effect.

Recently, an alternative to TENS, percutaneous stimulation, and bulky implantable stimulation assemblies has been introduced. Small, implantable microstimulators have been created that can be injected into soft tissues through a cannula or needle. However, these are not limited to only certain tissues. Internal organ systems cannot easily be reached with such techniques. Therefore, there is a need for using small, fully implantable, chronic neurostimulators for the purpose of treating chronic pain.

### General Therapy

### GERD Treatment:

Gastro-esophageal reflux disease (GERD) is a widespread affliction, which frequently elevates to be a clinical problem for the patient. It has been suggested that about ten percent of the U.S. population may have what is referred to as daily heartburn, and that more than one-third of the population has intermittent symptoms. Most therapies for GERD, which has a number of different manifestations, have historically been directed at neutralization or suppression of gastric acid. Although the use of antacid for self-medication of symptoms of GERD is prodigious, unfortunately many patients with mild esophagitis nonetheless progress to a more severe form of the disease.

While it is commonly said that the underlying problem that produces GERD is abnormal acid secretion, the literature suggests that in fact it is largely an esophageal motility disorder. By this it is meant that GERD is caused by abnormal motility which allows a breakdown of the anti-reflux barriers provided by the lower esophageal sphincter (LES) and esophageal-clearing peristalsis. The data point to decreased LES pressures in reflux patients. The more severe cases appear to be in patients having lower LES pressures with lower peristaltic amplitudes and abnormal peristalsis.

It is not clear whether the poor motility and low esophageal pressures of GERD patients precede esophageal mucosal reflux damage, or whether repeated reflux first results in a progressive decline in LES pressure. In any event, most patients with GERD who exhibit substantial esophageal injury also have abnormal LES pressures. One illustrative attempt to treat GERD with stimulation is shown in U.S. Pat. No. 5,716,385, Mittal et al. In that system, the skeletal muscles of the crural diaphragm are stimulated during relaxations of the diaphragm, causing contraction of the LES. However, this is a very indirect approach; the LES is not directly stimulated. Furthermore, the stimulation is applied only during sensed periods of transient relaxation.

By contrast, it is a premise of the system and method of this invention that therapy for GERD is best provided by substantially continuously increasing LES pressure. It is thus my concept to provide stimulation of the lower esophageal sphincter muscle to produce sustained and continuous contraction of the muscle so as to reduce acid reflux from the stomach. In other words, stimulation of the LES causes it to remain “tonal” or “excited,” so that it is “closed” to a sufficient degree to reduce acid reflux from the stomach whenever there may be significant output of gastric acid. The induced constriction of the lower esophageal sphincter by application of stimulus pulses to excite the sphincter muscle will reduce, and indeed can stop ongoing acid damage within the esophagus. By thus correcting the GERD condition, the patient will be relieved from having to rely on costly drugs or surgical procedures, neither of which is reliably effective. Such an implantable system can be used to continuously correct the problem of lower LES pressure. The system can therefore provide a reduction in the number of medical problems, e.g., esophagitis (inflammation of the lower esophagus); bleeding from the lower esophagus due to ulcerations caused by acid reflux; reducing the risk of stricture formation of the lower esophagus from acid injury; and formation of scar tissue due to natural bodily attempts to heal the damaged area. Further, reduction of reflux injury can lower the incidence of cancer of the lower esophagus. In patients who are at increased risk due to Barrett's esophagus, reduction of acid reflux is likely also to reduce the risk of subsequent cancer. Stimulation near one or more sites through a small entry point from the vascular system near a site such as lower esophageal sphincter may make the treatment with intravascular stimulation platform be effective.

### Neuronal Stimulation:

The autonomic nervous system (ANS) is the portion of the nervous system that controls the body's visceral functions, including action of the heart, movement of the gastrointestinal tract, and secretion by different glands, among many other vital activities, in order to maintain homeostasis of the body. The autonomic nervous system is linked and receives information from centers located in the spinal cord, brain stem, hypothalamus, and cerebral corte. xFurthermore, parts of the body send impulses by visceral reflexes into the centers in a dynamic, ongoing, multi-way dialogue, with each organ continuously influencing the other's function. This communication network is based along two major ways: neurological (through the transmission of nerve impulses) and biochemical (via hormones and neurotransmitters).

The two major subdivisions of the transmission system of the ANS (i.e., the sympathetic and parasympathetic) regulate the body in response to an ever-changing internal and external environment. The sympathetic system is known as the “body accelerator.” It activates the body and mind for exercise and work and it prepares the body to meet real or imagined threats to its survival. The parasympathetic system can be compared to a “brake.” When the parasympathetic system is activated, we generally tend to relax and slow down. But each system can have inhibitory effects in some organs and excitatory effects in others. For example, the generally exciting sympathetic system inhibits the digestive musculature and by exciting the microvascular arteriolar sphincters, reduces the digestive blood flow. Conversely, the enervating parasympathetic system is extraordinarily exciting for the digestive system, and increases the visceral blood circulation.

Pacing of the stomach and other portions of the gastrointestinal (GI) tract via electrical pulses has been experimented with for some time. Most of the experimentation has been oriented toward improving the gastric emptying usually by attempting to speed up the transit time of food moving through the GI tract (for failure to thrive, gastroparesis, or pseudo-obstruction) or of relieving the neurally mediated symptoms associated with gastroparesis. Stimulation near one or more sites through a small entry point from the vascular system near a site such as lower esophageal sphincter may make the treatment with intravascular stimulation platform be effective.

### Obstructive Apnea Treatment:

Obstructive Sleep Apnea (OSA) is a common disorder in western society, affecting between approximately 4% to 9% of the general population over the age of 40. It is a condition where the upper airway may be occasionally obstructed, either partially or completely, during sleep. Such obstructions may result in an interruption of sleep or at the least diminished quality of sleep. The primary clinical symptom is daytime hypersomnolence. This condition can significantly interfere with a patient's ability to function normally. Long-term medical consequences of chronic, untreated OSA may include pulmonary and systemic hypertension, cardiac arrhythmias, increased likelihood of myocardial infarction and ultimately, cardiac failure.

To treat obstructive sleep apnea, upper airway collapse can be relieved in many ways. One approach is to bypass the upper airway so that even if the airway collapses, there is an alternative route for air to flow. Such a bypass is accomplished through a tracheostomy procedure. This of course is highly invasive, costly and not currently favored. Another approach is to reverse the upper airway collapse. Many treatments may be used to reverse the upper airway collapse, including weight loss, pharmacological management, upper airway reconstructive surgery, or continuous positive airway pressure (CPAP). CPAP at present is now the most favored method for treating OSA, being used in approximately 80% of all newly diagnosed cases of OSA. In spite of its current widespread use CPAP is still not the ideal treatment. For example less than half of CPAP patients use CPAP regularly. More conservative measures such as weight loss and pharmacological treatment have also met with minimal success due to compliance problems or the development of side effects. Surgical reconstruction of the upper airway (uvulopalatopharyngoplasty or UPPP) has also met with equivocal results, mostly due to an inability to select the optimal patient for this particular form of treatment.

Stimulation of the upper airway and in particular of the hypoglossal nerve in synchrony with the inspiratory phase of respiration is a further alternative therapy for patients with OSA. Patients treated with such a upper airway stimulation system are provided the opportunity to gain restful, uninterrupted sleep otherwise not possible due to the obstructive apnea episodes. Such a system is available from Medtronic, Inc. The system for stimulation consists of an implanted programmable pulse generator, such as the Medtronic Inspire Model 3024 implantable pulse generator, a stimulating lead, e.g. the Medtronic Model 3990 half cuff electrode, and a dP/dt pressure sensing lead to signal respiration, such as the Medtronic model 4322 pressure sensor. Preliminary results demonstrate that hypoglossal nerve stimulation for treatment of OSA is successful.

In spite of the initial success, stimulation synchronized with respiration is, in some patients, a problem due to cardiac artifact in the pressure signal. Although in some patients the pressure signal is only minimally affected by the cardiac artifact, resulting in excellent synchronized pacing, in other patients cardiac artifact makes detection of respiration less reliable.

There is a need for an apparatus to sense airway blockage, monitor the blockage for a nominal time and stimulate hypoglossal nerve would be suitable for the apnea treatment.

### Therapeutic Stimulation of Muscles and Nerves:

Neuromuscular stimulation (the electrical excitation of nerves and/or muscle to directly elicit the contraction of muscles) and neuromodulation stimulation (the electrical excitation of nerves, often afferent nerves, to indirectly affect the stability or performance of a physiological system) and brain stimulation (the stimulation of cerebral or other central nervous system tissue) can provide functional and/or therapeutic outcomes. While existing systems and methods can provide remarkable benefits to individuals requiring neuromuscular or neuromodulation stimulation, many limitations and issues still remain. For example, existing systems often can perform only a single, dedicated stimulation function.

A variety of products and treatment methods are available for neuromuscular stimulation and neuromodulation stimulation. As an example, neuromodulation stimulation has been used for the treatment of urinary incontinence. Urinary incontinence is a lower pelvic region disorder and can be described as a failure to hold urine in the bladder under normal conditions of pressure and filling. The most common forms of the disorder can arise from either a failure of muscles around the bladder neck and urethra to maintain closure of the urinary outlet (stress incontinence) or from abnormally heightened commands from the spinal cord to the bladder that produce unanticipated bladder contractions (urge incontinence).

There exist both external and implantable devices for the purpose of neuromodulation stimulation for the treatment of urinary urge incontinence. The operation of these devices typically includes the use of an electrode placed either on the external surface of the skin, a vaginal or anal electrode, or a surgically implanted electrode. Although these modalities have shown the ability to provide a neuromodulation stimulation with positive effects, they have received limited acceptance by patients because of their limitations of portability, limitations of treatment regimes, and limitations of ease of use and user control.

Implantable devices have provided an improvement in the portability of neuromodulation stimulation devices, but there remains the need for continued improvement. Implantable stimulators described in the art have additional limitations in that they are challenging to surgically implant because they are relatively large; they require direct skin contact for programming and for turning on and off. In addition, current implantable stimulators are expensive, owing in part to their limited scope of usage.

These implantable devices are also limited in their ability to provide sufficient power which limits their use in a wide range of neuromuscular stimulation, and limits their acceptance by patients because of a frequent need to recharge a power supply and to surgically replace the device when batteries fail.

More recently, small, implantable microstimulators have been introduced that can be injected into soft tissues through a cannula or needle. Although these small implantable stimulation devices have a reduced physical size, their application to a wide range of neuromuscular stimulation application is limited. Their micro size extremely limits their ability to maintain adequate stimulation strength for an extended period without the need for frequent recharging of their internal power supply (battery). Additionally, their very small size limits the tissue volumes through which stimulus currents can flow at a charge density adequate to elicit neural excitation. This, in turn, limits or excludes many applications.

It is time that systems and methods for providing neuromuscular stimulation address not only specific prosthetic or therapeutic objections, but also address the quality of life of the individual requiring neuromuscular and neuromodulation stimulation.

There is a need for an apparatus that has adequate energy to be an effective therapy. The platform can also provide feedback for the stimulation efficacy for the neuromodulation stimulation.

### Skeletal Muscle Stimulation:

Two hundred thousand Americans are alive today who suffer from the chronic effects of spinal cord injury. Traumatic brain injury is the source of 500,000 hospitalizations every year in the United States, and each year 80,000 of these patients will retain a lifelong disability.

There are two general types of spinal cord injury: complete and incomplete lesions. Complete lesions leave the patient with no motor, sensory, or autonomic function below the level of the lesion. Transection of the spinal cord is the most obvious cause of a complete lesion. The level of the injury in the spinal cord determines exactly what function will be lost, as the spinal nerves which exit the cord below this are absolutely unable to transmit signals to or from the brain. Incomplete lesions can take a variety of forms, and depending on the nature of the trauma, a range of motor and sensory abilities may be present.

Additionally, non-traumatic pathologies such as stroke and Parkinson's disease are also often characterized by a patient's inability to successfully translate a desire to perform an action into the appropriate motions of the relevant limbs. In summary, central nervous system pathologies are often responsible for varying levels of paralysis which cause immense suffering in the affected population.

Rehabilitation efforts for these patients usually focus on teaching means for using still-functioning limbs to carry out desired tasks, while trying, when possible, to recover some function in the affected limbs. In addition, a range of technologically advanced, expensive, and—unfortunately—not very satisfactory devices have been built and tested on patients. Amongst these are muscle-stimulation devices, which include electrodes that are mounted on a patient's muscles in a paralyzed limb. In response to a command, the electrodes drive current into the muscles, causing the contraction thereof. The resultant motion of the limb is typically rough, and the unnatural stimulation protocols often leave the patient's muscles tired, even after performing only a small number of tasks.

What is now needed is an apparatus to control the stimulation device that has adequate energy to be an effective therapy. The apparatus should also provide feedback for the stimulation efficacy for the muscular stimulation.

### Pelvic Floor Stimulation:

Urinary incontinence affects millions of people, causing discomfort and embarrassment, sometimes to the point of social isolation. In the United States, recent studies have shown that as many as 25 million persons, of whom approximately 85% are women, are affected by bladder control problems. Incontinence occurs in children and young adults, but the largest number affected is the elderly.

There are several major forms of incontinence: Stress incontinence is an involuntary loss of urine while doing physical activities which put pressure on the abdomen. These activities include exercise, coughing, sneezing, laughing, lifting, or any body movement which puts pressure on the bladder. Stress incontinence is typically associated with either or both of the following anatomical conditions:

Urethral hypermobility—Weakness of or injury to pelvic floor muscles causes the bladder to descend during abdominal straining or pressure, allowing urine to leak out of the bladder. This is the more common source of stress incontinence.

Intrinsic sphincter deficiency—In this condition, the urethral musculature is unable to completely close the urethra or keep it closed during stress.

Urge incontinence is the sudden urgent need to pass urine, and is caused by a sudden bladder contraction that cannot be consciously inhibited. This type of incontinence is not uncommon among healthy people, and may be linked to disorders such as infections that produce muscle spasms in the bladder or urethra. Urge incontinence may also result from illnesses that affect the central nervous system.

Overflow incontinence refers to leakage of urine that occurs when the quantity of urine exceeds the bladder's holding capacity, typically as a result of a blockage in the lower urinary tract.

Reflex incontinence is the loss of urine when the person is unaware of the need to urinate. This condition may result from nerve dysfunction, or from a leak in the bladder, urethra, or ureter.

Of the major forms of incontinence listed above, the two most common are stress and urge. “Mixed incontinence” is a term used to describe the common phenomenon of the presence of stress and urge incontinence in the same patient.

A large variety of products and treatment methods are available for care of incontinence. Most patients suffering from mild to moderate incontinence use diapers or disposable absorbent pads. These products are not sufficiently absorbent to be effective in severe cases, are uncomfortable to wear, and can cause skin irritation as well as unpleasant odors. Other non-surgical products for controlling incontinence include urethral inserts (or plugs), externally worn adhesive patches, and drugs.

Exercise and behavioral training are also effective in some cases in rehabilitating pelvic muscles and thus reducing or resolving incontinence. Patients are taught to perform Kegel exercises to strengthen their pelvic muscles, which may be combined with electrical stimulation of the pelvic floor. Electromyographic biofeedback may also be provided to give the patients an indication as to the effectiveness of their muscular exertions. But retraining muscles is not possible or fully effective for most patients, particularly when there may be neurological damage or when other pathologies may be involved.

Medtronic Inc. produces a device known as InterStim®, for treatment of urge incontinence. InterStim® uses an implantable pulse generator, which is surgically implanted in the lower abdomen and wired to nerves near the sacrum (the bone at the base of the spine) in a major surgical procedure—sometimes six hours under general anesthesia. Electrical impulses are then transmitted continuously to a sacral nerve that controls urinary voiding. The continuous electrical stimulation of the nerve has been found to control urge incontinence in some patients.

Various surgical procedures have been developed for bladder neck suspension, primarily to control urethral hypermobility by elevating the bladder neck and urethra. These procedures typically use bone anchors and sutures or slings to support the bladder neck. The success rates for bladder neck suspension surgery in controlling urinary leakage are typically approximately 60%-80%, depending on the patient's condition, the surgeon's skill, and the procedure which is used. The disadvantages of this surgical technique are its high cost, the need for hospitalization and long recovery period, and the frequency of complications.

What is now needed is a device for treatment of both urinary stress incontinence and urge incontinence.

### Sacral Nerve Stimulation:

Pelvic floor disorders such as, urinary incontinence, urinary urge/frequency, urinary retention, pelvic pain, bowel dysfunction (constipation, diarrhea), erectile dysfunction, are bodily functions influenced by the sacral nerves. Specifically, urinary incontinence is the involuntary control over the bladder that is exhibited in various patients. Incontinence is primarily treated through pharmaceuticals and surgery. Many of the pharmaceuticals do not adequately resolve the issue and can cause unwanted side effects, and a number of the surgical procedures have a low success rate and are not reversible. Several other methods have been used to control bladder incontinence, for example, vesicostomy or an artificial sphincter implanted around the urethea. These solutions have drawbacks well known to those skilled in the art. In addition, some disease states do not have adequate medical treatments.

The organs involved in bladder, bowel, and sexual function receive much of their control via the second, third, and fourth sacral nerves, commonly referred to as S2, S3 and S4 respectively. Electrical stimulation of these various nerves has been found to offer some control over these functions. Several techniques of electrical stimulation may be used, including stimulation of nerve bundles within the sacrum. The sacrum, generally speaking, is a large, triangular bone situated at the lower part of the vertebral column, and at the upper and back part of the pelvic cavity. The spinal canal runs throughout the greater part of the sacrum. The sacrum is perforated by the anterior and posterior sacral foramina that the sacral nerves pass through.

Neurostimulation leads have been implanted on a temporary or permanent basis having at least one stimulation electrode positioned on and near the sacral nerves of the human body to provide partial control for bladder incontinence. Temporary sacral nerve stimulation is accomplished through implantation of a temporary neurostimulation lead extending through the skin and connected with a temporary external pulse generator.

In one embodiment, a lead bearing a distal stimulation electrode is percutaneously implanted through the dorsum and the sacral foramen (a singular foramina) of the sacral segment S3 for purposes of selectively stimulating the S3 sacral nerve. The lead is advanced through the lumen of a hollow spinal needle extended through the foramen, the single distal tip electrode is positioned adjoining the selected sacral nerve. Stimulation energy is applied through the lead to the electrode to test the nerve response. The electrode is moved back and forth to locate the most efficacious location, and the lead is then secured by suturing the lead body to subcutaneous tissue posterior to the sacrum and attached to the output of a neurostimulator IPG. Despite the suture fixation, sacral nerve stimulation leads having a single discrete tip electrode can be dislodged from the most efficacious location due to stresses placed on the lead by the ambulatory patient. A surgical intervention is then necessary to reposition the electrode and affix the lead.

The current lead designs used for permanent implantation to provide sacral nerve stimulation through a foramen have a number, e.g., four, ring-shaped, stimulation electrodes spaced along a distal segment of the lead body adapted to be passed into or through the foramen along a selected sacral nerve. Each distal stimulation electrode is electrically coupled to the distal end of a lead conductor within the elongated lead body that extends proximally through the lead body. The proximal ends of the separately insulated lead conductors are each coupled to a ring-shaped connector element in a proximal connector element array along a proximal segment of the lead body that is adapted to be coupled with the implantable neurostimulation pulse generator or neurostimulator IPG.

Again, the electrode array is moved back and forth with respect to the sacral nerve while the response to stimulation pulses applied through one or more of the electrodes is determined. The IPG is programmed to deliver stimulation pulse energy to the electrode providing the optimal nerve response, and the selection of the electrodes can be changed if efficacy using a selected electrode fades over time due to dislodgement or other causes.

Electrical stimulation pulses generated by the neurostimulator IPG are applied to the sacral nerve through the selected one or more of the stimulation electrodes in either a unipolar or bipolar stimulation mode. In one unipolar stimulation mode, the stimulation pulses are delivered between a selected active one of the stimulation electrodes and the electrically conductive, exposed surface of the neurostimulator IPG housing or can providing a remote, indifferent or return electrode. In this case, efficacy of stimulation between each stimulation electrode and the neurostimulator IPG can electrode is tested, and the most efficacious combination is selected for use. In a further unipolar stimulation mode, two or more of the stimulation electrodes are electrically coupled together providing stimulation between the coupled together stimulation electrodes and the return electrode.

In a bipolar stimulation mode, one of the distal stimulation electrodes is selected as the indifferent or return electrode. Localized electrical stimulation of the sacral nerve is effected between the active stimulation electrode(s) and the indifferent stimulation electrode.

A problem associated with implantation of permanent and temporary neurostimulation leads involves maintaining the discrete ring-shaped electrode(s) in casual contact, that is in location where slight contact of the electrode with the sacral nerve may occur or in close proximity to the sacral nerve to provide adequate stimulation of the sacral nerve, while allowing for some axial movement of the lead body.

Typically, physicians spend a great deal of time with the patient under a general anesthetic placing the leads due to the necessity of making an incision exposing the foramen and due to the difficulty in optimally positioning the small size stimulation electrodes relative to the sacral nerve. The patient is thereby exposed to the additional dangers associated with extended periods of time under a general anesthetic. Movement of the lead, whether over time from suture release or during implantation during suture sleeve installation, is to be avoided. As can be appreciated, unintended movement of any object positioned proximate a nerve may cause unintended nerve damage. Moreover reliable stimulation of a nerve requires consistent nerve response to the electrical stimulation that, in turn, requires consistent presence of the stimulation electrode proximate the sacral nerve. But, too close or tight a contact of the electrode with the sacral nerve can also cause inflammation or injury to the nerve diminishing efficacy and possibly causing patient discomfort.

It is generally desirable to minimize surgical trauma to the patient through surgical exposure of the tissue and sacrum and use of sutures or fixation mechanisms to hold the electrodes in place. It is preferred to employ a minimally invasive, percutaneous approach in a path extending from the skin to the foramen that the neurostimulation lead is extended through.

One such percutaneous approach involves implantation of a temporary neurostimulation lead that extends through the patient's skin and is attached to an external pulse generator. Typically, the external pulse generator and exposed portion of the lead body are taped to the skin to inhibit axial movement of the lead body. When a stimulation time period ends, the lead is removed through the skin by application of traction to the exposed lead body, and the incision is closed. The neurostimulation lead bodies are formed with surface treatment or roughening in a portion proximal to the neurostimulation electrode expected to extend from the foramen to the patient's skin that is intended to increase the resistance to unintended axial dislodgement of the lead body to stabilize the electrode. A length of the lead body is formed with indentations or spiral ridges or treated to have a macroscopic roughening. These surface treatments or geometries provide some acute fixation against the subcutaneous tissues, but they are necessarily insufficient to resist intentional retraction of the lead to remove it upon cessation of temporary stimulation.

What is now needed is an alternative safe method that overcomes many of afore mentioned shortcomings of the prior art methods.

### Molecular/Genetic/Drug Therapy Delivery:

According to the United States National Cancer Institute, approximately 4,000 specific conditions are known to be caused by genetic detects. The GeneMed Network states that each human being carries roughly a half dozen defective genes, and that about one in ten people have or will develop an inherited genetic disorder.

A composite of approximately 150,000 individual genes constitutes a human being. Variation in the structure of these genes can lead to disease. Many diseases are hereditively passed by a single gene, while many others are influenced by a collection of genes.

Several years ago, the Human Genome Project began mapping every human gene. The project is fostering an understanding of the very foundation of human disease and is enabling new therapies to treat and predict the onset of disease. One such therapy is gene therapy, which seeks to directly and beneficially modify the expression of genes through delivery of engineered genetic material. Foreign nucleotide sequences of either DNA or RNA are inserted into a patient's cells to result in either expression of non-integrated sequences or integration of sequences directly into the DNA of the cells.

Safe and efficient delivery of nucleotide sequences to appropriate cells poses one of the primary challenges to gene therapy. Vectors, which encapsulate therapeutic genes, have been developed to deliver the sequences. These vectors may be either viral or synthetic. Viral vectors, derived from viruses, are the primary vectors in experimental use today. Viruses efficiently target cells and deliver genome, which normally leads to disease. However, viral vectors for gene therapy are modified so that they may not cause disease. Rather, therapeutic recombinant genes are inserted into the vectors and delivered to target cells. Optimally, the modified viruses retain their ability to efficiently deliver genetic material while being unable to replicate.

Research in the field of gene therapy is still in the formative stages. Human trials only began in 1990 with ex vivo techniques, wherein a patient's cells were harvested and cultivated in a laboratory and incubated with vectors to modify their genes. Cells were then harvested and intramuscularly transplanted back into the patient. Trials quickly shifted to in vivo techniques, in which viral vectors are administered directly to patients, again intramuscularly. A variety of diseases are currently being evaluated as candidates for gene therapy, and a need exists in the art for improved vector delivery techniques.

While significant progress has been made, current gene therapy delivery techniques have many drawbacks. Viral vectors are inherently dangerous due to the innate ability of viruses to transmit disease. Furthermore, long-term effects of using viruses as delivery vehicles are unclear. Chances for error in modifying the viruses to vectors are significant, and consequences may be substantial, including potential irreversible alteration of the human gene pool. Also, delivery of the vectors to an efficacious portion of diseased cells has proven difficult and expensive.

Synthetic vectors have been developed to address the potential for disease transmission with viral vectors. These vectors are complexes of DNA, proteins, or lipids, formed in particles capable of efficiently transferring genes. However, synthetic vectors have thus far proved less effective than viral vectors and have been slower to gain acceptance.

Perhaps even more problematic than limitations of the vectors, intramuscular in vivo techniques, wherein vectors are delivered into a patient's muscle tissue, have proven somewhat ineffective in clinical use. Systemic expression of inserted sequences is not realistic since therapy is localized.

In view of the drawbacks associated with previously known methods for delivery of gene therapy, it would be desirable to provide methods and apparatus that overcome such drawbacks.

In addition to gene therapy techniques, research has focused on the selective implantation or injection of cells or specific proteins to mitigate disease states, cause tissue regeneration or improve organ function. For example, researchers have investigated improvement of cardiac function by injecting cells via epicardial, endocardial or coronary sinus access routes into the myocardium. Others have investigated injecting cells into the pancreas or liver to improve insulin production in diabetics. The injection of donor spleen cells from non-diabetic mice into diabetic mice is described in literature so that a protein complex secreted by the spleen cells could mitigate the autoimmune disorder causing diabetes. Others describe infusion of isolated islets of Langerhans into a patient to alleviate Type-I diabetes. Other prior art describes the infusion of isolated islet cells through a catheter and into a vein in a patient's liver following partial pancreatectomy, so that the islets graft onto and function similarly to the removed liver. Still others have discovered that certain proteins, such as apolipoprotein A-I Milano, when introduced into the rats fed a high cholesterol diet, inhibits the onset of arterial thrombus formation. In view of the foregoing, it further would be desirable to provide methods and apparatus for delivering cells, cell components or naturally-occurring or synthetic proteins into the vascular system of a patient to achieve a treatment goal.

It still further would be desirable to provide methods and apparatus for providing localized delivery of genes, cells or bioactive agents into a patient's vascular system that have a preselected residency beyond that obtainable by systemic or localized intravascular infusions.

It also would be desirable to provide methods and apparatus for delivering viral vectors, synthetic vectors, drugs, cells, or naturally-occurring or synthetic proteins or other therapeutic agents in a manner that nourishes and sustain production and secretion of the therapeutic agents in vivo.

What is now needed is to provide methods and apparatus for delivering bioactive agents intravascularly, wherein, once the efficacious agent has dispersed, the delivery system reconfigures to mitigate risk of complication to the patient.

### Intravascular Measurement of Parameters

### Pressure:

A cardiac stimulating apparatus is described in U.S. Pat. No. 6,026,324 that non-intrusively determines a value indicative of hemodynamic pulse pressure from an accelerometer signal obtained by an accelerometer sensor enclosed in an implantable casing of the stimulating apparatus. The accelerometer sensor is electrically coupled to a microprocessor based controller and the accelerometer transmits a signal to the controller associated with fluid and myocardial accelerations of the patient's heart. A filtering arrangement is coupled to the accelerometer for filtering and conditioning the signal transmitted by the accelerometer to produce a waveform related to a pulse pressure within the patient's heart. In order to remove ancillary information contained in the acceleration signal the signal is transmitted through a series of filters. Thus, the above-referenced United States patent discloses a device capable of non-intrusively (meaning that no sensor needs to be inserted into the heart) determines a waveform related to the pressure and in particular the pulse pressure within a patient's heart.

Measuring pressure inside a heart by inserting a pressure sensor into the heart is well-known in the art. One example is given in the background section of U.S. Pat. No. 6,026,324 where it is referred to U.S. Pat. No. 4,566,456 discloses a device that adjusts the stimulation rate relative to right ventricular systolic pressure. The ventricular systolic pressure is measured by a piezoelectric pressure sensor mounted on lead inserted into the heart, i.e. an intrusive pressure measurement technique.

In order to obtain accurate and reliable measurements of the intracardial pressure it is often preferred to perform pressure measurements by arranging a pressure sensor inside the heart.

Intracardiac pressure is a highly valuable parameter for estimation of cardiac condition and cardiac pumping efficiency. Technically there is no difficulty in placing a pressure sensor in e.g. the right ventricle of a heart.

Although the pressure sensor may give a correct picture of the pressure at the sensor site, however, the pressure measured in an active patient is a summation of pressures having different origins. Apart from the desired component i.e. the pressure originating from the heart's pumping action, the sensor signal will contain pressure components from other sources such as vibration, external and internal sounds and barometric pressure changes.

In this context, it is relevant to note, that an 11 meter elevation in air gives rise to a pressure change of 1 mm of Hg. Also, it should be noted that the blood column in the body (in the actual case mainly the blood column in the heart) generates pressure changes when the body is exposed to exercise and/or vibrations.

External and internal sounds also can make a non-negligible contribution to the pressure signal. Examples of such external sounds are traffic noise and loud music and internal sounds such as coughing, sneezing and snoring.

Taking the above into account, it is fairly difficult to extract the desired signal i.e. the pressure signal emanating solely from the heart's pumping action, from the sensor signal.

For many applications it would be sufficient to measure the cardiac pressure during limited time intervals. One issue is then how to find intervals during which the cardiac pressure signal is the dominating signal contributor.

What is now needed is a versatile framework for signal monitoring that further uses ECG trigger for intravascular pressure determination.

### Hemodynamic Parameters:

Hemodynamic parameters are measurable attributes associated with the circulatory system of a living body, such as, for example, blood flow rate, blood pressure, volume of the vasculature, volume of the cardiac chambers, stroke volume, oxygen consumption, heart sounds, respiration rate, tidal volume, blood gases, pH, and acceleration of the myocardium. There are numerous medical reasons for sensing and tracking changes in hemodynamic parameters, including the proper operation of implantable cardiac stimulation devices.

Implantable cardiac stimulation devices (such as pacemakers, defibrillators, and cardioverters) are designed to monitor and stimulate the heart of a patient who suffers from a cardiac arrhythmia. Using leads connected to the patient's heart, these devices typically stimulate the cardiac muscles by delivering electrical pulses in response to detected cardiac events which are indicative of a cardiac arrhythmia. Properly administered therapeutic electrical pulses often successfully reestablish or maintain the heart's regular rhythm.

Modern implantable devices have a great number of adjustable parameters that can be tailored to a particular patient's therapeutic needs. Any of a number of parameters that define pacing characteristics may be optimized. Adjustable parameters may include, for example, the atrio-ventricular (A-V) delay, the R-R interval, and the pacing mode (e.g. pace and sense in the ventricle, pace and sense in the atrium and the ventricle, etc.). As an example, the A-V delay is typically optimized in dual-chamber (atrial and ventricular) pacemakers to time the ventricular contraction such that the contribution of the atrial contraction is maximally exploited. As another example, ventricular synchronization may be optimized in biventricular pacing for heart failure by adjusting the timing at which pacing pulses are delivered to various cardiac sites.

Typically, interchamber pacing intervals (such as A-V delay in dual chamber pacemakers and RV-LV delay in biventricular pacemakers) are set to default nominal values, or else relatively labor-intensive methods are used to measure hemodynamic variables in an effort to optimize some or all of the parameters at the time a cardiac stimulation device is implanted. Examples of measurements that may be carried out in connection with device programming include ultrasound to measure mitral flow and/or ejection fraction and left heart catheterization to measure the rate of change of left ventricular pressure during systole, which is a measure of contractility and mechanical efficiency.

One common technique for setting device parameters involves manually varying the operating parameters of a pacing system while monitoring one or more physiological variables. Typically, the optimum value for a parameter is assumed to be that which produces the maximum or minimum value for the particular physiological variable. This manual method can be time-consuming, during which the underlying physiologic substrate may change and give rise to inaccurate assessment of cardiac performance. Additionally, the manual method is prone to errors occurring during data gathering and transcription.

An automated technique for setting at least one type of device parameter entails systematically scanning through a series of available A-V pulse delays at a fixed heart rate while monitoring a measure of cardiac output, then setting the A-V pulse delay to the value which resulted in the maximum cardiac output. Another technique selects the A-V pulse delay by maximizing the measured value (e.g. by electrical impedance) of a parameter such as stroke volume.

Another method for automatically selecting a cardiac performance parameter entails periodically pacing the heart for a short period of time with stimulating pulses having a modified pacing parameter value, and then allowing the heart to return to a baseline value for a relatively long time. The cardiac performance parameter is monitored both during and after the heart is paced to determine if it has improved, degraded, or remained the same. The heart is then paced with a modified pacing parameter value and the process is repeated.

The optimization of pacing parameters is not necessarily critical in patients with relatively normal myocardium, although it may be beneficial to them. These patients have the necessary cardiac reserve to compensate for programming errors. It is patients with depressed cardiac function that are much more sensitive to factors such as pacing rate and A-V delay. Current optimization techniques are time-consuming and labor intensive. Furthermore, they are prone to error because they do not account for variability in the measured hemodynamic signals that often obscures real and significant changes in hemodynamic status and complicates measuring the absolute values of hemodynamic parameters. What is now needed is a method or system for detecting changes in hemodynamic parameters as well as obtaining accurate absolute measurements of such parameters.

### Glucose:

Diabetes mellitus is a serious medical condition affecting millions of Americans, in which the patient is not able to maintain blood glucose levels within the normal range (normoglycemia). Approximately 10% of these patients have insulin-dependent diabetes mellitus (Type I diabetes, IDDM), and the remaining 90% have non-insulin-dependent diabetes mellitus (Type II diabetes, NIDDM). The long-term consequences of diabetes include increased risk of heart disease, blindness, end-stage renal disease, and non-healing ulcers in the extremities.

The continuous in vivo monitoring of glucose in diabetic subjects should greatly improve the treatment and management of diabetes by reducing the onus on the patient to perform frequent glucose measurements. Implanted glucose sensors could be used to provide information on continuously changing glucose levels in the patient, enabling swift and appropriate action to be taken. In addition, daily glucose concentration measurements could be evaluated by a physician. An implantable sensor could also provide an alarm for hypoglycemia, for example, overnight, which is a particular need for diabetics. Failure to respond can result in loss of consciousness and in extreme cases convulsive seizures. Similarly, a hyperglycemic alarm would provide an early warning of elevated blood glucose levels, thus allowing the patient to check blood or urine for ketone bodies, and to avert further metabolic complications.

There are two main approaches to the development of a continuous blood glucose monitor. The first category is non-invasive sensors, which obtain information from physical-chemical characteristics of glucose (spectral, optical, thermal, electromagnetic, or other). The second category is invasive sensors. In this group, there is intimate mechanical contact of the sensor with biological tissues or fluids, since the device is placed within the body. Invasive glucose sensors may be categorized based on the physical principle of the transducer being incorporated. Current transducer technology includes electrochemical, piezoelectric, thermoelectric, acoustic, and optical transducers. It should be noted that most diabetes patients have concomitant heart conditions such as CHF and small artery disease.

### Localized Drug Delivery:

Various medical devices have been developed for the delivery of therapeutic agents to the body. However, many challenges remain in providing drugs at desired target sites for sustained lengths of time.

For example, the problem of vascular injury presents a significant challenge during balloon angioplasty and coronary stenting procedures. Unfortunately, a limited number of controlled, long term, localized drug delivery systems have been developed that can address the complications of vascular injury, for example, endothelial denudation and exposure of the highly thrombotic subendothelial layer. Although some medical devices such as drug-coated stents provide a vehicle for sustained localized delivery of therapeutic agents (e.g., immunosuppressive and/or antiproliferative agents), other medical devices such as balloon angioplasty devices do not.

Drug delivery nanocapsules comprise (a) a drug-containing core and (b) a polyelectrolyte multilayer encapsulating the drug-containing core. Such nanocapsules can be prepared, for example, using various known layer-by-layer (LbL) techniques. LbL techniques typically entail coating particles, which are dispersed in aqueous media, via nanoscale, electrostatic, self-assembly using charged polymeric (polyelectrolyte) materials. These techniques exploit the fact that the particles serving as templates for the polyelectrolyte layers each has a surface charge, which renders them water dispersible and provides the charge necessary for adsorption of subsequent layers (i.e., polyelectrolyte multilayer encapsulation). The charge on the outer layer is reversed upon deposition of each sequential polyelectrolyte layer. Such multilayer shells are known to provide controlled drug release. For example, shell properties such as thickness and permeability can be tuned to provide an appropriate release profile.

Numerous materials, such as proteins, have an inherent surface charge that is present on particles made from the same. Examples of charged polymeric therapeutic agents include polynucleotides (e.g., DNA and RNA) and polypeptides (e.g., proteins, whose overall net charge will vary with pH, based on their respective isoelectric points), among others. For example, insulin is a negatively charged molecule at neutral pH, while protamine is positively charged.

Other materials, for example, many solid and liquid organic compounds, are uncharged. Such materials, however, can nonetheless be encapsulated by LbL technique by (a) providing the compound in finely divided form using, for instance, (i) colloid milling or jet milling or precipitation techniques, to provide solid particles, or (ii) emulsion technique to provide liquid particles within a continuous liquid or gel phase. The particles are provided with a surface charge, for example, by providing least one amphiphilic substance (e.g., an ionic surfactant, an amphiphilic polyelectrolyte or polyelectrolyte complex, or a charged copolymer of hydrophilic monomers and hydrophobic monomers) at the phase boundary between the solid/liquid template particles and the continuous phase (typically an aqueous phase).

Once a charged template particle is provided, it can be coated with a layer of an oppositely charged polyelectrolyte. Multilayers are formed by repeated treatment with oppositely charged polyelectrolytes, i.e., by alternate treatment with cationic and anionic polyelectrolytes. The polymer layers self-assemble onto the pre-charged solid/liquid particles by means of electrostatic, layer-by-layer deposition, thus forming a multilayered polymeric shell around the cores.

Traditional techniques just use the properties of nanoparticles to provide localized drug delivery. While this effective in some cases, there are cases where there is need to provide localized drug delivery over a prolonged period of time wherein a predetermined amount of nanoparticles could be released at predetermined times. Therefore, there is a need for an apparatus for powering and programmatically controlling the delivery of nanoparticles, whether they are stored in a container or in a pump.

### Molecular Therapy Delivery:

There are several molecular therapies that can be delivered using the wireless intravascular platform described in this invention. In an exemplary embodiment, stimulation of angiogenesis without causing a contractile muscle response is described. Specifically, a low-voltage electrical stimulation of skeletal muscle induces synthesis of new VEGF protein and promotes angiogenesis. Essentially this embodiment involves a subthreshold device and more efficient methods for the controlled delivery of angiogenic growth factors to promote angiogenesis in muscle tissue, and methodologies that can be used to stimulate angiogenesis in cardiac and vascular tissue.

The key step of the embodiment is applying “voltage” stimulation of a prespecified threshold, to activate a number of proteins, which in turn switch on angiogenesis. The information needed to enable this may include: the knowledge of an exact protein being targeted and the related specific voltage band for this targeted protein; and the knowledge of proteins that may have similar molecular weight and electrical specificity that may result in incidental activation. If, however, all that was looked for was “general activation,” then the specific relationships between the targeted protein and the activation voltage range may not be required.

Thus it is clear for the foregoing detailed description that a platform for providing integrated implanted healthcare is of great importance and meets a significant void in the currently available approaches for such an approach.

### SUMMARY OF THE INVENTION

The invention provides an intravascular implantable system to provide electrical stimulation of a tissue for a purpose to deal with a clinical condition in an animal. The system comprises a power supply module supplying energy to the implantable system; an implanted control module controlling the functioning of the implantable system and initiating desired digital waveforms wherein the envelope of the waveform is a predetermined attribute; an implanted intravascular sensing module sensing at least one parameter of interest for the purpose to deal with the clinical condition; and an intravascular stimulation module electrically stimulating the tissue with a output waveform that is substantially similar to the desired digital waveform initiated by the control module.

The power supply module may use an implantable non-rechargeable battery, or an implantable rechargeable battery, or a wireless energy source based on a near-field resonant, inductive coupling.

The implanted intravascular sensing module uses at least one parameter of interest related to: pressure, volume, flow, electrical, mechanical, thermal, chemical, electrolyte level, position, location, glucose level, urea level, drug delivery, oxygen concentration, carbon dioxide level, measure of blood thinning and drug level.

The intravascular implantable system is well suited for various clinical applications of electrical stimulation including therapy monitoring, detection or sensing of evoked responses, therapy and treatment.

Various clinical conditions that can be treated include: irregular cardiac rhythms, slow or fast cardiac rhythms, infarct repair, ischemia detection, tachycardia stimulation/cardiac stimulation, chronic heart failure resynchronization, seizure prevention, seizure warning, obsessive compulsive disorder, spine problem, obstructive airway disorder, neuronal disorder, GERD, gastro-intestinal disorder, endo tracheal problem, skeletal muscle problem, pelvic floor problem, sacral nerve problem, depression, obesity, pain relief, nerve damage, pancreatic disorder, chronic constipation problem, and internal wounds.

The intravascular implantable system can be used to stimulate tissue from various organs such as brain, heart, esophagus, stomach, kidney, ear, eye, lung, uterus, prostate, blood, spine, bladder, pancreas, colon and nerve.

The digital stimulation waveform is intermittent, interrupt driven or event driven for the contemplated applications.

### BRIEF DESCRIPTION OF DRAWINGS

FIG. 1 is a representation of and intravascular medical device is used as a cardiac pacing system attached to a medical patient;

FIG. 2 is an isometric, cut-away view of a patient's blood vessels in which a receiver antenna, a stimulator and an electrode of the intravascular medical device have been implanted at different locations;

FIG. 3A is a schematic of an exemplary wireless intravascular platform for tissue stimulation illustrating external and internal components;

FIG. 3B is a block schematic diagram of the exemplary wireless intravascular platform illustrating extravascular and intravascular components;

FIG. 4A is a schematic diagram of part of the controller providing a three-state output;

FIG. 4B is the table showing the relationship between signal state and the output;

FIG. 4C is an exemplary signal waveform;

FIG. 4D is the table showing the relationship between output signals and logical states corresponding to the signal waveform in FIG. 4C;

FIG. 4E is an exemplary analog amplifier;

FIG. 4F depicts the losses incurred during driving the analog amplifier;

FIG. 4G shows a pulse width modulated signal in a unipolar option;

FIG. 4H shows a pulse width modulated signal in a bipolar option;

FIG. 4I illustrates decoding an arbitrary desired waveform that is encoded by PWM signal via a biological filter;

FIG. 4J shows a desired digital waveform going through a biological filter and retaining its final shape;

FIG. 5 is a illustrates the application landscape of the generalized wireless intravascular platform;

FIG. 6 schematically depicts an alerting system enabled by the wireless intravascular platform;

FIG. 7 is a block schematic diagram of a monitoring system enabled by the wireless intravascular platform;

FIGS. 8A and 8B are schematic diagrams of an sensing amplifier system enabled by the wireless intravascular platform;

FIGS. 9-12 are schematic representations of various attributes of a detection system enabled by the wireless intravascular platform;

FIG. 13 is a block schematic diagram of a classification method enabled by the wireless intravascular platform;

FIG. 14A depicts a circuit of a high impedance lead in a prior art system;

FIG. 14B shows the circuit of the system enabled by the proposed method;

FIG. 15 is the representation of one period of a standard pulse;

FIG. 16 is the representation of one period of one form of the proposed composite pulse;

FIG. 17 is the representation of one period of an alternative form of the proposed composite pulse;

FIG. 18 is a schematic of the arterial stimulation from an adjacent vein;

FIG. 19 is a schematic of the hybrid treatment system enabled by the wireless intravascular platform;

FIG. 20 is a schematic of the energy supply and control signal provided by the wireless intravascular platform to an implanted sensing and monitoring device;

FIG. 21 is a schematic of the energy supply and control signal provided by the wireless intravascular platform to an implanted sensing and treatment/therapy device;

FIG. 22 is a schematic of the energy supply and control signal provided by the wireless intravascular platform to an implanted sensing and monitoring device; and

FIG. 23 is a schematic of the energy supply and control signal provided by the wireless intravascular platform to an implanted supporting device.

### DETAILED DESCRIPTION OF THE INVENTION

Although the present invention is being described in the context of implanted components of a cardiac pacing system, it can be used in the implanted components for other types of medical devices in an animal's body. Furthermore, the present apparatus and method are not limited to implanted items in a therapy providing system, but can be employed to implanted elements for other purposes in the animal as described in subsequent paragraphs.

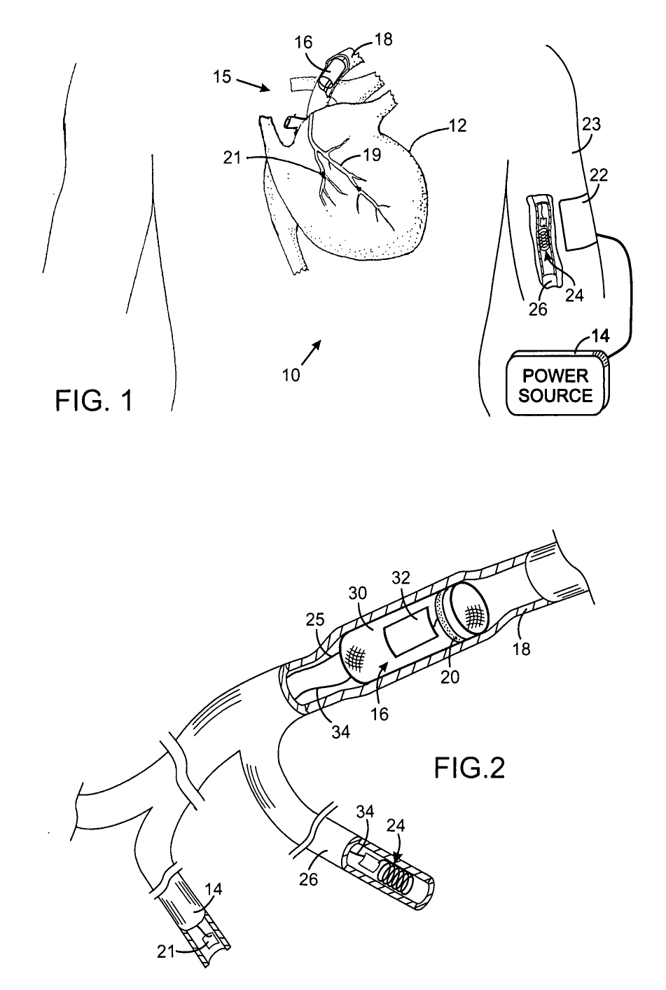

Initially referring to FIG. 1, a cardiac pacing system 10 for electrically stimulating a heart 12 to contract comprises an external power source 14 and a medical device 15 implanted in the circulatory system of a human medical patient. The medical device 15 receives a radio frequency (RF) signal from the power source 14 worn outside the patient and the implanted electrical circuitry is electrically powered from the energy of that signal. At appropriate times, the medical device 15 delivers an electrical stimulation pulse into the surrounding tissue of the patient.

The power source 14 may be the same type as described in U.S. Pat. Nos. 6,445,953 and 6,907,285 and includes a radio frequency transmitter that is powered by a battery. The transmitter periodically emits a signal at a predefined radio frequency that is applied to a transmitter antenna in the form of a coil of wire within an adhesive patch 22 that is placed on the patient's upper arm 23. In a basic version of the cardiac pacing system 10, the radio frequency signal merely conveys energy for powering the medical device 15 implanted in the patient. In other systems, the transmitter modulates the radio frequency signal with commands received from optional circuits that configure or control the operation of the medical device 15.

Referring to FIGS. 1 and 2, the exemplary implanted medical device 15 includes an intravascular stimulator 16 located a vein or artery 18 in close proximity to the heart. Because of its electrical circuitry, the stimulator 16 is relatively large requiring a blood vessel that is larger than the arm vein, e.g. the basilic vein, which is approximately five millimeters in diameter. Therefore, the stimulator 16 may be implanted in the superior or inferior vena cava. However, it is contemplated that miniaturization of components can allow the electrical circuitry needed to be much smaller the example cited above. Electrical wires lead from the stimulator 16 through the cardiac vascular system to one or more locations in smaller blood vessels 19, e.g. the coronary sinus vein, at which stimulation of the heart is desired. At such locations, the electrical wire 25 are connected to a remote electrode 21 secured to the blood vessel wall.

Because the stimulator 16 of the medical device 15 is near the heart and relatively deep in the chest of the human medical patient, a receiver antenna 24 for the RF signal is implanted in a vein or artery 26 of the patient's upper right arm 23 at a location surrounded by the transmitter antenna within the arm patch 22. That arm vein or artery 26 is significantly closer to the skin and thus receiver antenna 24 picks up a greater amount of the energy of the radio frequency signal emitted by the power source 14, than if the receiver antenna was located on the stimulator 16. Alternatively, another limb, neck or other area of the body with an adequately sized blood vessel close to the skin surface of the patient can be used. The receiver antenna 24 is connected to the stimulator 16 by a micro coaxial cable 34.

As illustrated in FIG. 2, the intravascular stimulator 16 has a body 30 constructed similar to well-known expandable vascular stents. The stimulator body 30 comprises a plurality of wires formed to have a memory defining a tubular shape or envelope. Those wires may be heat-treated platinum, Nitinol, a Nitinol alloy wire, stainless steel, plastic wires or other materials. Plastic or substantially nonmetallic wires may be loaded with a radiopaque substance which provides visibility with conventional fluoroscopy. The stimulator body 30 has a memory so that it normally assumes an expanded configuration when unconfined, but is capable of assuming a collapsed configuration when disposed and confined within a catheter assembly, as will be described. In that collapsed state, the tubular body 30 has a relatively small diameter enabling it to pass freely through the vasculature of a patient. After being properly positioned in the desired blood vessel, the body 30 is released from the catheter and expands to engage the blood vessel wall. The stimulator body 30 and other components of the medical device 15 are implanted in the patient's circulatory.

The body 30 has a stimulation circuit 32 mounted thereon and, depending upon its proximity to the heart 12, may hold a first electrode 20 in the form of a ring that encircles the body. Alternatively, when the stimulator 16 is relatively far from the heart 12, the first electrode 20 can be remotely located in a small cardiac blood vessel much the same as a second electrode 21. The stimulation circuit 32, which may be the same type as described in the aforementioned U.S. patents, includes a power supply to which the micro coaxial cable 34 from the receiver antenna 24 is connected. The power supply utilizes electricity from that antenna to charge a storage capacitor that provides electrical power to the stimulation circuit. A conventional control circuit within the stimulation circuit 32 detects the electrical activity of the heart and determines when electrical pulses need to be applied so that the heart 12 contracts at the proper rate. When stimulation is desired, the stimulation circuit 32 applies electrical voltage from its internal storage capacitor across the electrodes 20 and 21. The second electrode 21 and the first electrode when located remotely from the stimulator 16, can be mounted on a collapsible body of the same type as the stimulator body 30. In all the examples cited with regard to the FIG. 2, it should be understood that the example size limit is driving the decision on the placement of components. It is contemplated that miniaturization of components can lead to many more options for component placement.

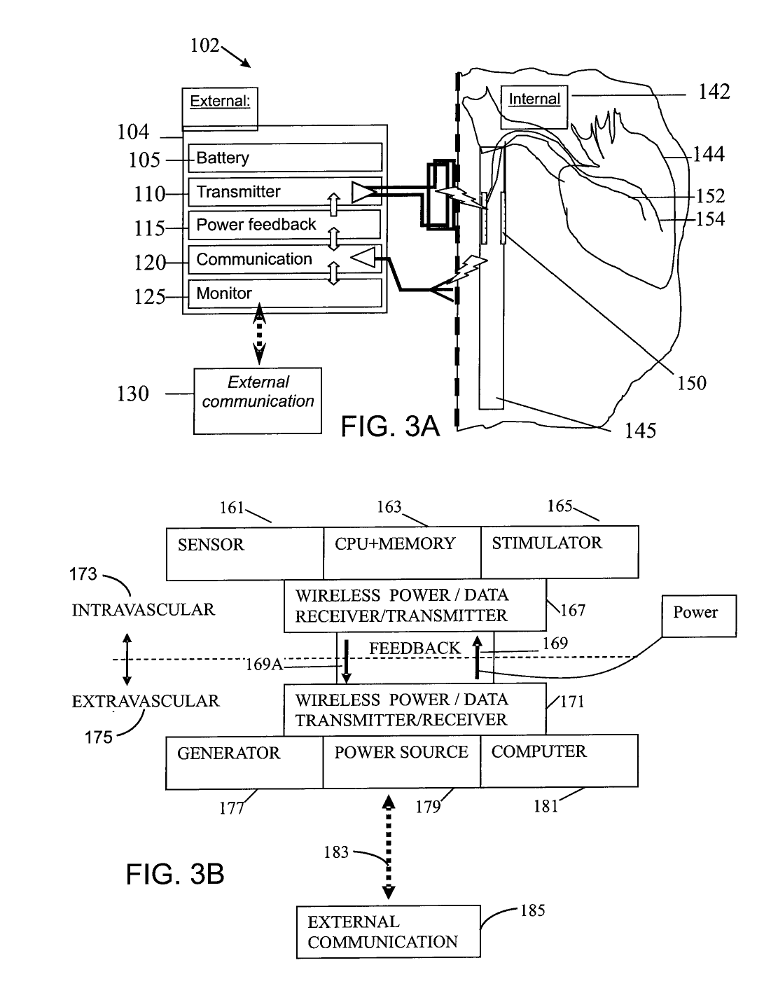

FIG. 3A shows the schematic of a wireless intravascular platform 102 for tissue stimulation illustrating external components 104 located outside the body of an animal and internal components 142 located inside the body of the animal. The external components 104 include a battery 105, power transmitter 110, power feedback module 115, a communication module 120 and a monitor 125. The external components may optionally include a wireless communication module 130 to communicate with external devices (not shown).

Battery 105 is rechargeable allowing for patient mobility with periodic recharge cycles. With battery volume, the time between recharge cycles can be proportioned to cover days, months or years. Power transmitter 110 is a modulated transmitter proportioned to provide maximum power with an adjustable duty cycle to meet the power demands. Power feedback module 115 is part of closed loop system composed of power transmitter, implanted component 150 comprising of an RF receiver coil and an electronics capsule and a feedback algorithm to supply a required amount of power. The control loop converts the receiver voltage into a frequency shift of the secondary re-transmitter. Consequently, a drop in received voltage would cause an increase in the retransmitted frequency. (E.g. on a 100 MHz signal, this would be a 10 to 50 kHz shift per 100 mV). Since the power consumption is a function of the number of pacing events, the power level itself could vary. By maintaining a constant voltage, it is ensured that only the needed amount of power is transmitted.

Communication module 120 receives logged data collected from the implant device. This data can be physiological data and a set of trending logs indicating patient and/or device condition over time. Trending logs can be accumulated continuously by the receiver CPU by keeping the highest time resolution for the most recent events in minutes, the mid-range events hours, and long range events in days etc. Alternatively, the logged data can have a fixed size, wherein the actual storage of data can be done externally. Internally, since the CPU has a limited space, one may choose to maintain the most recent data at a higher time resolution. As another alternative, the data from the implant can be streamed in real-time to an external storage and the externally stored data can be analyzed for the trends. In one embodiment, for reporting purposes, one could extract data around events, e.g. time prior to the detection of arrhythmias, and time after pacing attempts to restore the rhythm. For the purposes of patient management, for instance, the data from the implant could alert the physician when conditions requiring quick follow up such as atrial fibrillation requiring anticoagulation occurred. Other physiological parameters such as change in blood volumes, heart rate variability, pressure changes, and blood sugar can be used for short and long term trending for internal monitoring and alerting.

The communication module 120 also provides an access point into the system to communicate to the caregiver or to alert a caregiver remotely by means of auto-dialup, for example, in case an alerting condition presents itself. Monitor 125 monitors the received data.

Again referring back to FIG. 3A, the internal components 142 include the implanted component 150 mentioned above consisting of an RF receiver coil and an electronics capsule located in a large vessel 145. One example of such a vessel is inferior vena cava (IVC). In one exemplary embodiment leads 152 and 154 are used for pacing and sensing the heart 144 respectively.

### The Generalized Wireless Intravascular Platform

The generalized form of the wireless intravascular platform described above is summarized in FIG. 3B. It has both an intravascular component 173 and an extravascular component 175. The extravascular component may be implanted or extracorporeal.

### Power Supply:

The core of the platform consists of a power source 179 that is extravascularly located and employs wireless transmission of power to operate the intravascular platform. It has a computer 181 that is used to perform a number of functions including overall control logic, processing algorithms, data and power encoding and determination of optimal response based on the feedback. A signal generator 177 is associated when needed with the extravascular part of the platform to send data to the intravascular component 173 via a wireless power-data transmitter/receiver 171.

A discriminator circuit may be used to separate power and data components transmitted from extravascular component. The received power can be used for the intravascular operation by rectifying the power signal into DC by a rectification circuit and used to power the internal control and other electrical/electronic circuitry. Alternatively, the received power can be used to charge a rechargeable battery based on the need and used to meet the energy demands of the intravascular platform. In some embodiments, a combination of the above may be used for meeting the energy demands. In some embodiments, the extravascular component may have a non-rechargeable battery that powers the intravascular component. In some embodiments, the extravascular component may have a rechargeable power supply that may be charged by resonant, near-field inductive coupling, which is described below.

The main aspect of the power supply is an implanted resonant receiver coil which is inductively coupled to the input power source. A resonant receiver coil permits a higher collected energy density for a given receiver coil volume. In a resonant receiver coil, the induced voltages and currents are much higher than in a non resonant coil. As a result, a resonant coil with a given dimension and a high quality resonant circuit can collect more energy from a surrounding near-field than a non-resonant coil. A coil can be made resonant by adding a capacitor in parallel to create a parallel resonant circuit, or in series to create a series resonant circuit. The apparent impedance of the resonant circuit depends on the resistive loading on that tank circuit. The loading may be direct or indirect. In the case of a direct load, the load is placed directly across the resonant circuit. If the load is a linear resistor, it will have a dampening effect to lower the Q-value of the tank circuit and potentially nullify the benefit from the resonance. In the case of an indirect load, the load can be inductively or capacitively coupled externally. A load of this type is body tissue or blood pool.

Second, special precautions are taken to extract energy from the resonant circuit without excessive damping. For example, lowering the Q from 40 to 20 may be acceptable. However lowering the Q from 40 to less than 5 may not be. By using a capacitively coupled rectifier and using the rectifier to charge a buffer capacitor, the load is only presented to the resonant circuit when the rectifier is conducting. The time constant of the buffer capacitor and the load is chosen to allow, for example, a 1% droop in voltage between charge pulses. This effectively makes the load to appear only during the top 1% of the cycle. After initial charge-up, all that needs to be supplemented by the resonant circuit is at nearly full amplitude within the 1% mentioned in the exemplary case. The supplemented power is provided by a power feedback as previously described.

By combining these two aspects described above, an efficient energy source can be created. One additional aspect to consider is the transfer efficiency factor. Note that direct short wiring is the most efficient energy transfer with lowest resistance. For the wireless circuits, resonant coupled circuits are the most efficient with a high coupling factor when the primary (source) and the secondary (load) are next to each other with minimal space as in a near field scenario. In this case, the captured flux increases in a non-linear fashion. The resonant aspect focuses on a narrow band of the energy spectrum. The resonant energy has alternating electric fields coexistent with alternating magnetic fields. The energy may be derived from either one, as the fields are just a description of the two measurable aspects of the electromagnetic field transfer. However, the power dissipation in biological tissue is determined by the square of the electric field times the conductivity of the tissue divided by the density of the tissue for the computation of specific absorption rate (SAR). Therefore, the preferred energy transfer mechanism is via the B field. Antennas are designed such that E field is minimized. It should be noted that there are two types of electric fields: one is caused by varying magnetic field as described by Maxwell's equations, and will always be there. The other is caused by voltage sources. It is the latter aspect of the electric field that is minimized by the choice of magnetic field antennas. Hence these antennas are loops that carry current and generate magnetic field.

The extravascular component 175 communicates via a link 183 with an external device 185.

### Controller:

A controller 163 controls the stimulation signal with a digital output delivered to the stimulation site. The control circuit stores the operational parameters for use in controlling operation of a stimulator that applies tissue stimulating segmented voltages pulses across a plurality of electrode pairs. Preferably, the control circuit comprises a conventional microcomputer that has analog and digital input/output circuits and an internal memory that stores a software control program and data gathered and used by that program. The controller also controls an electrical sensing device that does not have external grounding or referencing. The sensing device and the controller are connected to the tissue through a lead assembly with a plurality of dynamically programmable electrodes, which may or may not be shared with the pacing electrodes. Purpose specific segmented waveforms are delivered to the electrodes by the controller. The controller may be located at an intravascular location or located at a suitable subcutaneous location. The controller generates desired digital stimulation waveform.

A signal receiver/transmitter 167, when needed, may also function as a stimulator as in the exemplary embodiment described previously in FIGS. 2 and 3A. Both of the wireless power/data transceivers may be linked by feedback loops 169 in one direction and 169A in the other direction that may optimize the power and data transfer between the transceivers. The receiver/transmitter 167 may be located at an intravascular location and may receive signals from the waveform generator using a wireless means. As in case of the example above, the reception may be from a near-field, resonant inductive coupling. Stimulator 165 when needed may be located at a suitable intravascular location. It has stimulation leads that may be directly wired to the receiver or to the waveform generator. Sensors 161 when needed may be located at a suitable intravascular location in one embodiment as shown or they may be located subcutaneously (not shown). Sensors may be active requiring power from the power supply to operate or passive requiring no additional power from the external or an internal power source. Sensing leads when needed may be located at an intravascular location. Alternatively, they may be located at a subcutaneous location. In certain cases, the sensing leads may be connected to a generator directly. Alternatively the connection may be indirect, for example, through a resonant, near-field, inductive coupling. In some embodiments, electrodes and sensing leads may terminate in the vessel they are deployed. In this case, stimulation and sensing may be carried out in a transvascular manner. In some embodiments, electrodes and/or sensing leads may exit the vessel they are deployed through an opening in the vessel wall and may be directly anchored to the tissue to be stimulated and/or sensed from. In some embodiments, electrodes and/or sensors may be freely suspended in the blood steam of the vessel. The generalized form of the wireless intravascular framework described above can be used for several applications that will be described in detail below.

### Waveform Synthesis:

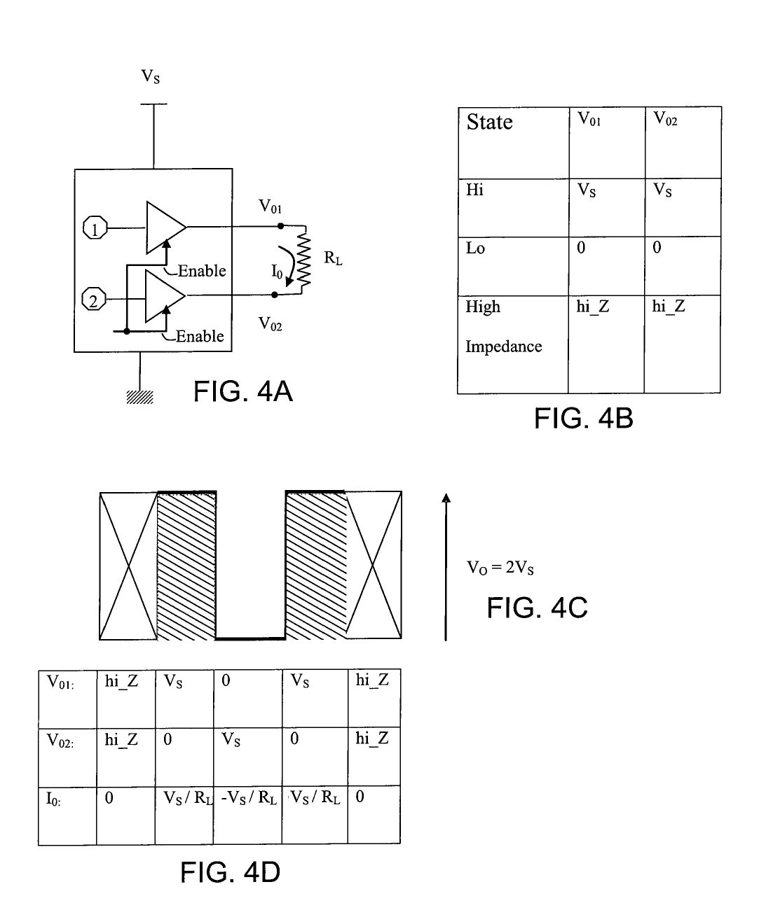

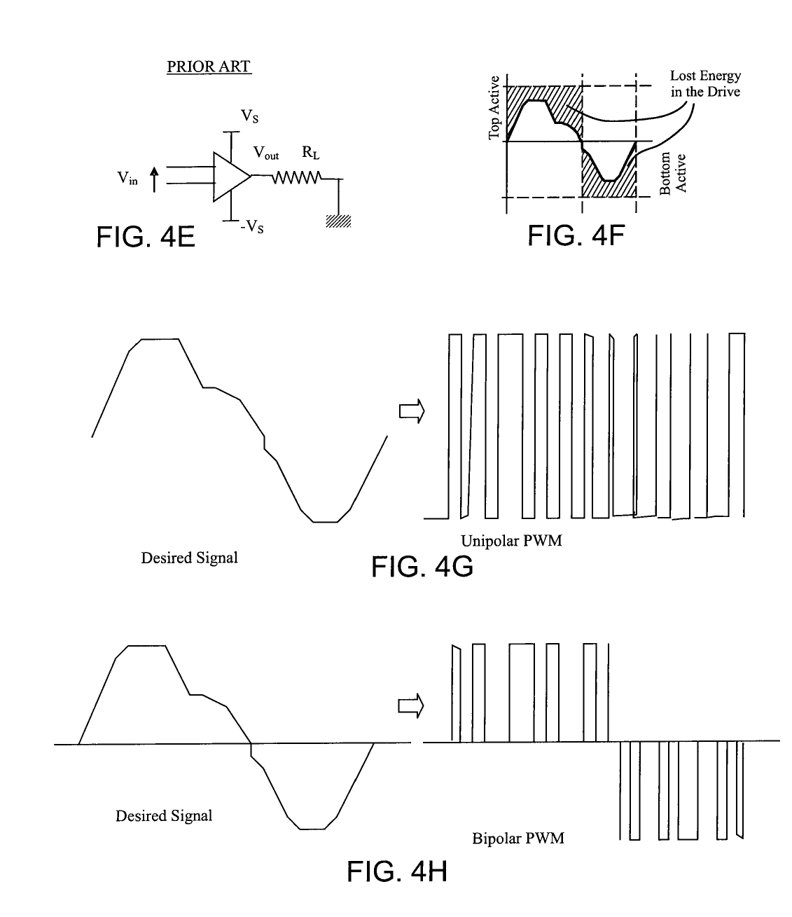

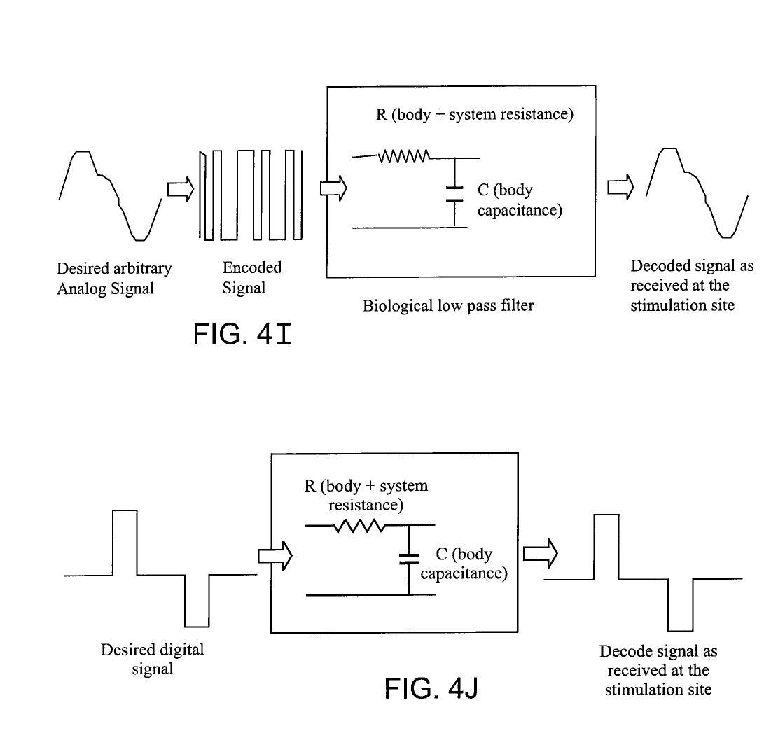

As mentioned before, the controller has multiple roles. In this section, the role of controller in synthesizing waveforms for stimulation is described. The FIG. 4A shows a portion of the controller delivering a generic digital output, which has a tri-state mode. The “enable” function is used to “turn-on” the outputs such that they can produce logic state high or a logic state low. In these states the current can be “sourced” from high (supply rail, VS) to the output, or “sinked” from output to ground. In the third state, i.e., the high impedance state, the output current is always zero. Thus it provides infinite impedance. This is shown in FIG. 4B.

The output derived from two such output voltages now gives a differential output, or a difference between these two outputs. The output voltage relative to common level (Vcm) is not relevant since it may be subtracted out as shown below:

V₀₁=Vcm+V_{0-A}

V₀₂=Vcm+V_{0-B}

V₀=V₀₁−V₀₂=V_{0-A}−V_{0-B}

If V_{0-A} and V_{0-B} can only be “0” or “VS” or open, then the composite or differential V₀ can produce VS, −VS, 0, or open. Note that an “open” voltage is not equal to “0.” In FIG. 4C, an exemplary waveform is shown and its various voltages, logical states and output current through a load resistance RL are shown in FIG. 4D. Note that there is no reference to a system common or the external ground anywhere. By using the difference of two signals that each can be in one of three states, a multitude of waveform envelopes can be synthesized. This synthesis may not be possible by using the ground referenced single ended signals.

A conventional analog output which is shown in FIG. 4E. Note that the conventional analog output is inherently not energy efficient. This is indicated in FIG. 4F by highlighting area representing energy loss around the arbitrary waveform.

The envelope of the synthesized waveform may be determined or selected as a function of measurement a physiological characteristic which is sensed by an implanted and/or an external sensor. The sensed characteristic may be a naturally occurring or an evoked in response to the electrical stimulation from the implanted medical device. In one embodiment, externally sensed motion may be used in conjunction with an internally sensed heart rate to provide an adaptive algorithm for waveform envelope selection. In another embodiment, the envelope of the synthesized waveform is a function of a command signal transmitted to the implanted device via RF telemetry. In a further embodiment, the synthesized waveform's envelope is a function of preprogrammed clinical algorithm that may be application dependent. Such a preprogrammed clinical algorithm may be needed in an emergency care situation, for example. In a general case, the envelope of the synthesized waveform may be a function of an attribute that can be a sensed signal, received telemetry signal, or a preprogrammed clinical algorithm to mention only a few.

In a digital system, FIGS. 4G and 4H show how arbitrary waveforms can be encoded using digital output. The first one shown in FIG. 4G is a unipolar pulse width modulation (PWM) waveform that may be used for the case of a differential output. The second one shown in FIG. 4H is a bipolar PWM for the case of ground referenced outputs wherein the center line is the ground reference. The encoded signal is similar to naturally occurring neural firings into a muscle, which are PWM as well. There are two methods of PWM. The first one has fixed pulse width and variable frequency similar to the coding occurring in a neural system. Alternatively, one may use variable pulse width with fixed frequency.

FIG. 4I shows a simplified schematic of signal application to the tissue. The pulse width modulated signal is applied to the tissue. The body impedance characteristics are conveniently used as a biological filter. The resultant integration comes from the body tissue resistance combined with the natural tissue capacitances. The biological integrator, i.e., low-pass filter system, smoothes out the ripple thereby reconstructing/decoding the stimulation signal substantially similar to the desired signal synthesized by the controller. It should be noted that the resistance R of the low pass filter system includes both the body resistance and the system resistance. Since body resistance is constant but low, the system resistance needs to be substantially lowered compared to the traditional electrodes to minimize the distortion of the waveform including rounding of the corners and keep it substantially similar to the desired waveform.

Having described a general case, the preferred embodiment is described below. In the preferred embodiment, the controller delivers segmented digital waveforms whose voltage envelope is chosen such that it is close to the desired output voltage. In such a device capture threshold is managed by modifying the duration of the output waveform to minimize energy losses at the output stage. In this case as shown in FIG. 4J, since the waveform is already in the digital form, one may not need to use PWM. As mentioned before, the system resistance needs to be substantially lowered compared to the traditional electrodes to minimize the distortion of the waveform and keep it substantially similar to the desired waveform. The segmented, stimulation waveforms may pass through a voltage intensifier stage based on a specific purpose. As an example of an application requiring voltage intensification stage, an atrial defibrillation treatment device may require a high voltage (10-30 volts) and high rate of 1200 beats/minute (BPM). As an example for an application that does not require voltage intensification, a pacing device to treat bradycardia may need a low voltage (2-5 volts) and low rate (40-120 beats/minute).

### APPLICATIONS

### Application Paths:

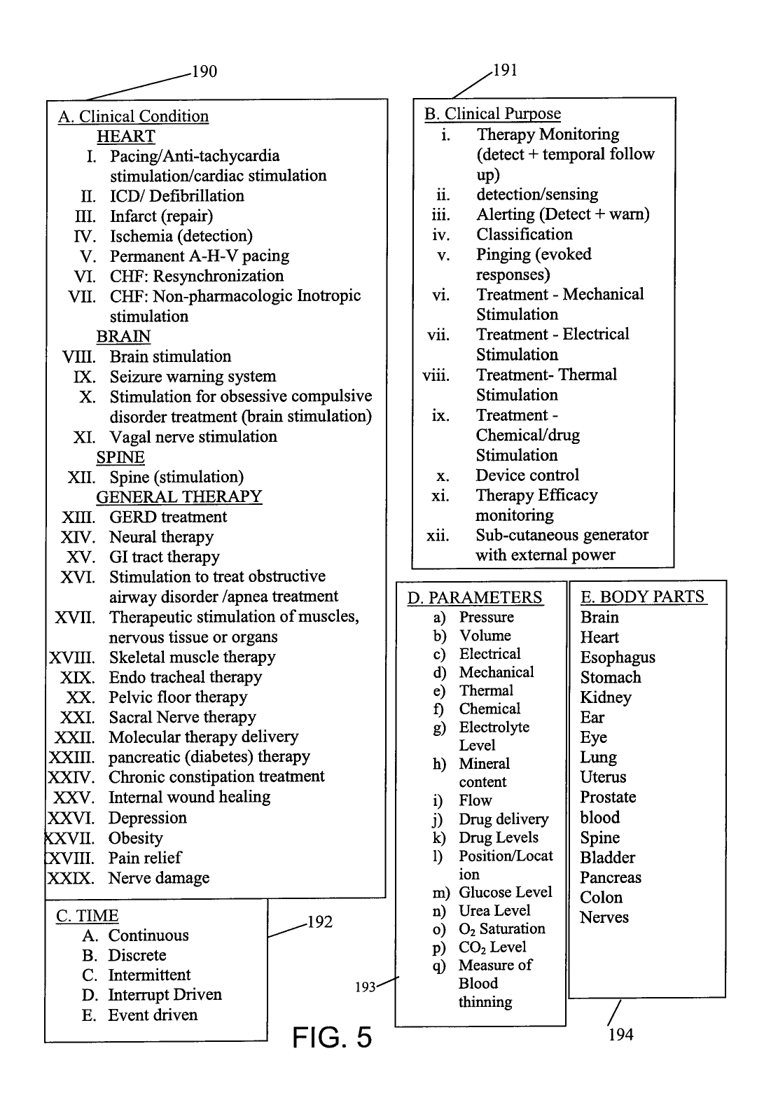

Using a wireless intravascular platform in its generalization, several application paths may be defined. FIG. 5 illustrates a matrix of potential application paths definable and contemplated by the aspects of wireless intravascular platform. Each contemplated application path may include one or more components of one or more of an attribute of a clinical condition (A) 190, a clinical purpose attribute (B) 191, a temporal attribute (C) 192, a parameter attribute (D) 193 and a body part attribute (E) 194. These sub-components may form a series of non-mutually exclusive listings. However, not all of the paths possible by the listings are novel. Rather, specific paths adapted for specific care purposes and specific body parts using the wireless intravascular platform (WIVP) are contemplated. The combinations of WIVP application paths are constructed with at least one of the attributes of A, B, C and D in the following combinations. Accordingly, wireless intravascular platform for attributes A, B, A+B, B+C, A+C, A+D, B+D, C+D, A+B+C, B+C+D, A+C+D, A+B+D, A+B+C+D are contemplated. Here the “+” notation is to indicate the Boolean “AND” operation. These paths are further explained in the examples below.

Wireless intravascular platform for A: Wireless intravascular platform for treating at least one clinical condition; examples: wireless intravascular platform for CHF resynchronization therapy; and wireless intravascular platform for CHF therapy involving non-pharmacologic inotropic stimulation.

Wireless intravascular platform for B: Wireless intravascular platform for a clinical purpose; Example: Wireless intravascular platform for CHF resynchronization therapy monitoring.

Wireless intravascular platform for B “AND” A: Wireless intravascular platform for a clinical purpose attribute to act on a clinical condition attribute; Example: Wireless intravascular platform for monitoring patients during CHF resynchronization therapy.

Wireless intravascular platform for C “AND” B: Wireless intravascular platform for an implanted device control. Example: Wireless intravascular platform for an event triggered insulin pump control.

Wireless intravascular platform for C “AND” A: Wireless intravascular platform for temporal attribute manipulating an implanted device in response to a clinical condition. Example: Wireless intravascular platform for an event triggered cardiac pacing for CHF patients.

Wireless intravascular platform for D “AND” B: Wireless intravascular platform for parameter attribute manipulating a clinical purpose to treat a patient. Example: Wireless intravascular platform for remote monitoring of electrical parameters of chronic cardiac failure patients.

Wireless intravascular platform for C “AND” D: Wireless intravascular wireless platform for temporal attribute to modulate parameter attribute. Example: Wireless intravascular platform for continuous intravenous pressure measurement.

Wireless intravascular platform for C “AND” D with B: Wireless intravascular wireless platform for temporal attribute to modulate a parameter attribute to achieve a clinical purpose (i.e., one or multi-parameter and one or multi-purpose). Example: Wireless intravascular platform for periodically alerting a caregiver on a patient's vital signs.

Wireless intravascular platform for C “AND” D with A: Wireless intravascular wireless platform for a temporal attribute to modulate a parameter attribute to treat a clinical condition. Example: Wireless intravascular platform for periodically monitoring electrolyte levels during GI tract stimulation.

Wireless intravascular platform for D with B and A: Wireless intravascular platform for a parameter value driven therapy modification. Example: Wireless intravascular platform for cardiac signal (ECG) based electrical stimulation treatment of CHF.

Wireless intravascular platform for B on A with C: Wireless intravascular platform for a clinical purpose attribute to act on a clinical condition attribute using a temporal attribute. Example: Wireless intravascular platform for deep brain stimulation for epileptic seizures based on temporal EEG signal analysis.

Wireless intravascular platform for B on A with C and D: Wireless intravascular platform for a clinical purpose attribute to act on a clinical condition attribute using a temporal attribute in conjunction with a parameter attribute. Example: Continuous glucose monitoring to control the amount of electrical stimulation for chronic obesity treatment.

Wireless intravascular platform for multiple B: Wireless intravascular platform for therapy convergence. Example: Wireless intravascular platform to perform a number of treatment options including electrical stimulation treatment and drug treatment.

As illustrated with the last scenario, it should be understood that two or more options within the same attribute, B in the above scenario, can also be combined in a desired application.

### Alerting:

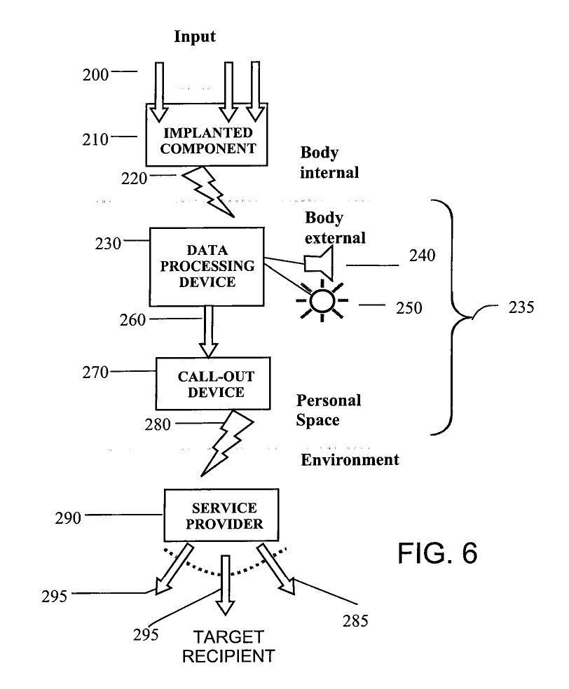

FIG. 6 schematically illustrates functional aspects of the alerting system enabled by the intravascular platform. The alerting system comprises several sub-components. The first sub-component, which is located inside the body of an animal, is the data input component 200 that collects processed and/or unprocessed data for physiological monitoring and system performance. An example for the system performance parameters that could be monitored is the energy transfer efficiency, which would be zero if the external component 102 shown in FIG. 3 were removed from the patient. The data is initially accessed by a computer in the implanted component 210 and is communicated to the external device through wireless means 220 for processing by the external component 235.

The second sub-component, which is part of the external component, is a data processing device 230, interpreting the presented data and comparing these to preset or programmable thresholds, which include static or dependant variables, such as rates that change with time. For example, a threshold could be the maximum allowable change of the heart rate. Another example of a variable can be the energy consumption over time.

The third sub-component is one or more of the communication devices that could be one or more of the following: audio means 24) for generating sounds at various levels; display means 250 for generating simple lights to text or waveforms or video or combination displays; and audio and voice using pre-recorded messages, associated with conditions and/or measurements. For example, an audio signal could indicate when an optimal relative position of external and internal components has not been achieved. In this example, a user can reposition the external component to minimize the audio signal, which falls below the audible range when optimal relative position is achieved. A second example could be a message indicating that the device should be repositioned on the patient. The message stops when the device is properly repositioned.

The fourth component is auto-communicator 270 that interface via signal path 260 with the data processing device 230. In one example, it could be a portion of a cell phone or a comparable wireless means 280 that calls a responsible party, a target recipient 295, that may be a caretaker, a primary physician, or a relative. This call may be directly to the target recipient or through a service provider 290. The wireless means may also call for service 285 in case of a device break down or when service is required. In another example, an acoustical warning emanated from the system, in the form of a subtle beep to prompt the user to activate the message system, which can be initiated in a suitable location to provide privacy if needed. In another example, the alerting mechanism can activate sound, or light if the device is dislodged and no longer with the patient. This alert enables localization of the external component and assist retrieve the component. In the same example, if no action takes place for a pre-determined time, the next level of alerting can be initiated.

In yet another example, if the data indicates either a serious condition of the patient, for example, absence of heartbeat for a pre-specified amount of time, or failure to attach the device, an automatic call can be placed using conventional existing cell phone networks. A prerecorded message, along with physiological data, where applicable, can be transmitted to the target audience. The prerecorded message can also be accessed by the target audience when their designated pager is activated.

It should be noted that the data transfer may not be a scheduled data transfer, but rather an impromptu situation-based, autonomous communication to allow corrective action at a tiered level, commensurate to the situation. In autonomous operation, the device will take action based on a set of criteria and circumstance. In some embodiments, environmental variables, such as air pressure, air temperature and skin temperature may be incorporated to correlate with physiological data prior to a communication decision being made.

As previously noted, the communication signal could be one or more of the following in the form of a low level audible alarm, an escalated audible alarm, a dial out, a dial out and voice exchange—as in comparable cell phone function, a data exchange or a multi-media data exchange.

With regards to the intravascular implanted system, it is capable of self monitoring, physiological monitoring and autonomously alerting the patient, a bystander, a remote expert, a networked computer, a service person or a relative. Thus it is further intended to include alerting mechanism to communicate with different, independent communicable targets based on both the needs of the device and the patient based on pre-determined conditions. In a first example, a caretaker can be alerted if internal and external components do not communicate with each other for a predetermined time. In a second example, the alerting mechanism may contact a medical service or physician if abnormal rhythms are observed. In a third example, the alerting mechanism may trigger a service call if communication is present but battery power is lower than a predetermined value.

### Monitoring:

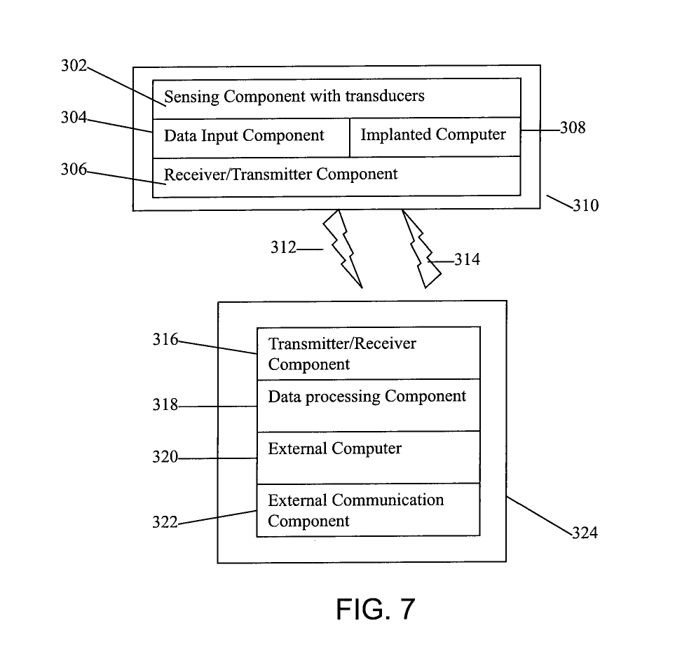

FIG. 7 schematically illustrates functional aspects of the monitoring system enabled by the intravascular platform of FIG. 4. The monitoring system involves sensing a physiological event and following it in time. The monitoring system mainly consists of intravascular component 310 and an extravascular component 324. Both these components may have several sub-components.

Now referring to he component 310, the first sub-component, which is located inside the body of an animal, is the sensing component 302 that senses the physiological parameter via one or more transducers. For example, the sensors of the present invention may be employed to provide measurements of volume. flow rate, pressure, temperature, electrical parameters, biochemical characteristics, or the amount and type of deposits in the lumen of an intravascular implant, such as a stent or other type of intravascular conduit. The present invention also provides a means to modulate mechanical and/or physical properties of the intravascular implant in response to the sensed or monitored parameter. Quantitative in vivo measurements of volumetric flow rate, flow velocity, biochemical constitution, fluid pressure or similar

physical or biochemical property of the body fluid through an intravascular device would provide more accurate diagnostic information to the medical practitioner. As used herein, the term “intravascular device” is intended to include stents, grafts and stent-grafts which are implanted within an anatomical passageway or are implanted with a body to create a non-anatomical passageway between anatomically separated regions within the body. The term “sensor,” as used herein, includes, without limitation, biosensors, chemical sensors, electrical sensors and mechanical sensors. While the term “biosensor” has been used to variously describe a number of different devices which are used to monitor living systems or incorporating biological elements, the International Union for Pure and Applied Chemistry (IUPAC) located in Research Triangle Park, N.C., U.S.A. has recommended that the term “biosensor” be used to describe “a device that uses specific biochemical reactions mediated by isolated enzymes, immunosystems, tissues, organelles or whole cells to detect chemical compounds usually by electrical, thermal or optical signals.” The term “chemical sensor” is defined by the IUPAC as a device that transforms chemical information, ranging from concentration of a specific sample component to total composition analysis, into an analytically useful signal. Conventional biosensors are a type of chemical sensor that consists of three basic elements: a receptor (biocomponent), transducer (physical component) and a separator (membrane or coating of some type). The receptor of a chemical sensor usually consists of a doped metal oxide or organic polymer capable of specifically interacting with the analyte or interacting to a greater or lesser extent when compared to other receptors. In the case of a biosensor the receptor or biocomponent converts the biochemical process or binding event into a measurable component. Biocomponents include biological species such as: enzymes, antigens, antibodies, receptors, tissues, whole cells, cell organelles, bacteria and nucleic acids. The transducer or physical component converts the component into a measurable signal, usually an electrical or optical signal. Physical components include: electrochemical devices, optical devices, acoustical devices, and calorimetric devices as examples. The interface or membrane separates the transducer from the chemical or biocomponent and links this component with the transducer. They are in intimate contact. The interface separator usually screens out unwanted materials, prevents fouling and protects the transducer. Types of interfaces include: polymer membranes, electropolymerized coatings and self-assembling monomers.

The second sub-component is the data input component 304 that collects processed and/or unprocessed data for physiological monitoring and system performance. An example for the system performance parameters that could be monitored is the energy transfer efficiency, which would be zero if the external component 102 shown in FIG. 3 were removed from the patient. The data is initially accessed by a computer in the implanted component 308 and is communicated to the external device through a wireless means, e.g. a receiver/transmitter component 306, for processing by the external component 324. By their nature, implantable sensors must have some mechanism for communicating sensed information from the sensor to a reader, which may be human or machine, outside the body. Since it is impractical to implant a physical connection between the sensor and the external reader, alternative means for generating a readable signal external the body is provided. Suitable means for generating a readable signal external the body include, without limitation, radiographically visible signals, magnetic flux signals, chemical signals, chemifluorescent signals, and/or electromagnetic signals. In a specific embodiment, radio frequency means can be used for wireless communication between sensor and an external device.

Now referring to the extravascular component 324, it may have a transceiver component 316 for bidirectional communication 312 and 314 with intravascular component 310. It may also have a data processing component 318 for interpreting the presented data and comparing these to preset or programmable thresholds, which include static or dependant variables, such as rates that change with time. For example, a threshold could be the maximum allowable change of the heart rate. Another example of a variable can be the energy consumption over time. The data processing component may be a part of the external computer 320 or it may have a self-contained computing capability with its own memory and logic. Additionally, the extravascular component may have an external communication sub-component 322 for communicating with a programmer. It may also be used with the alerting system described in FIG. 6.

### Sensing:

The description of parameter sensing is described in the monitoring section above and is incorporated herein by reference. While any of the sensing transducers adapted for use with the intravascular platform may be used, in the following, a signal amplifier and associated electronics that does not require a separate ground is described. The rationale for the signal amplifier is that in an implanted system, an additional ground line will only see common mode especially when the signal pair is either from a coaxial or a twisted pair.

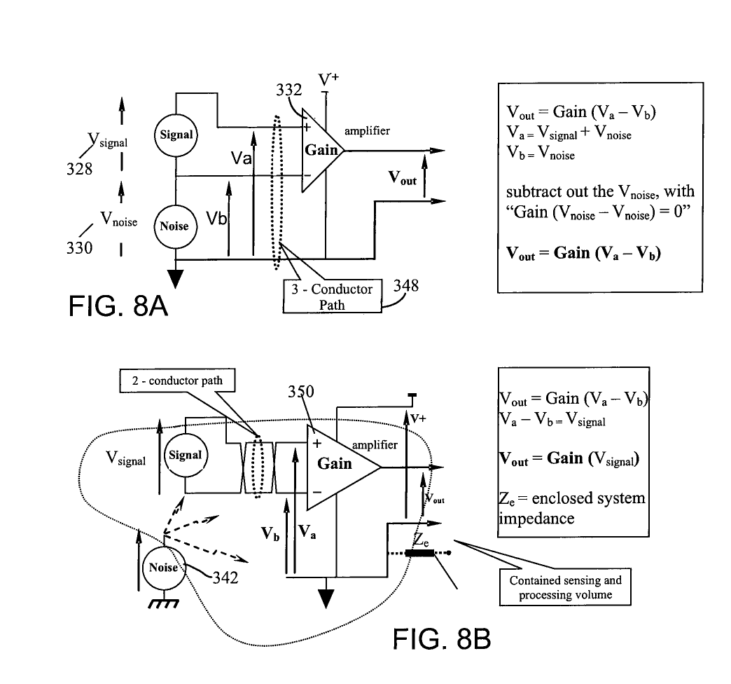

In an exemplary case of electrical sensing and amplifying of physiological signals is shown in FIG. 8A, wherein, the amplifier 332 has competing electromagnetic signal sources that may cause deterioration of signal quality performance. Established methods include the use of common mode rejecting amplifier designs, which reference the leads of a signal pair 328 to a reference, a real or virtual ground. When the signals have amplitudes in the range of few tens of mV, the performance of such solutions is good, as the operating voltage range is many orders of magnitude greater than the supplied signal, thereby allowing for large “common mode” signals to be superimposed on the signal of interest.

If the main signal leads, providing Va and Vb are contained within a space or volume with noise sources external to that volume as would be the case in an implanted system, the reference or ground lead may be removed with a concomitant performance improvement of the system as shown in FIG. 8B. By removing the external reference or ground, the signal lines maybe exposed to common mode noise. However, without a path to reference this noise, a common mode circuit cannot be formed resulting in the original signals to be presented to the amplifier. By arranging the two signal leads in a twisted pair fashion 348, it can be ensured that input conductor impedance for the signal amplifier 350 is equal for both the leads with equal noise exposure.

Noise voltage 342 can still be injected within each individual conductor and present an unbalanced noise component to the amplifier 350 where it will be amplified and spoil the original signal. Depending on location and application, the contributions of unbalanced noise must be considered before choosing this method.

In the FIG. 7B, Ze 354 is a virtual component, representing the impedance to the enclosing volume 346. When the enclosing volume has low impedance to the noise generator, it will form an electrostatic shield, whose effectiveness increases proportionally to the conductivity of that environment.

### Detection and Classification:

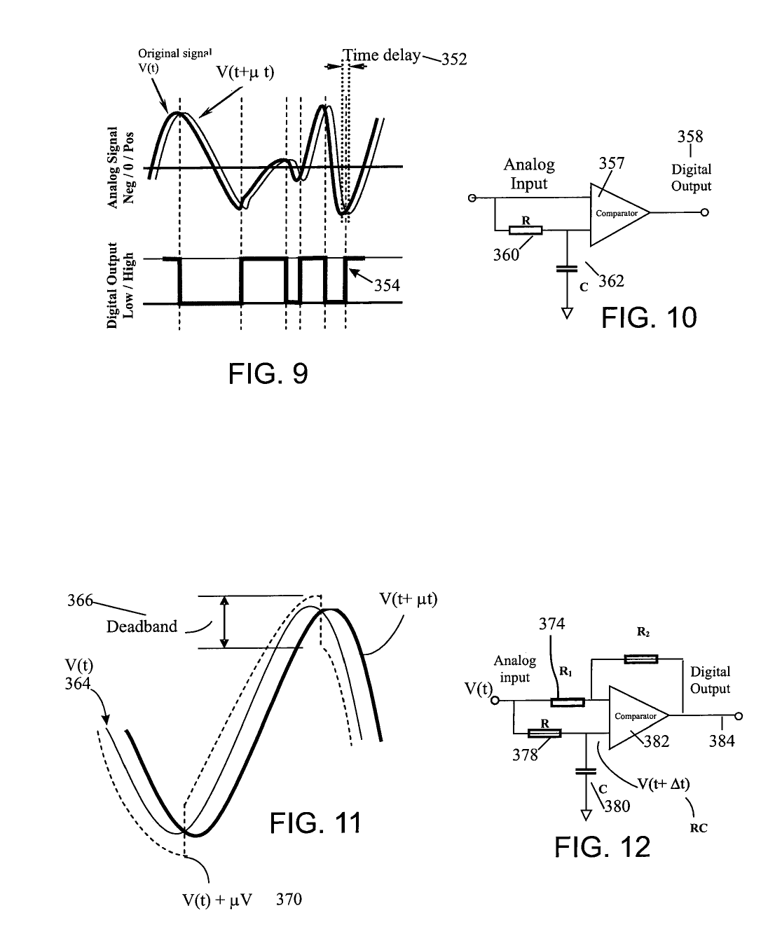

FIGS. 9, 10, 11 and 12 schematically illustrate functional aspects of the detection system enabled by the intravascular platform. The detection can be done during the signal acquisition, post signal acquisition or during both.

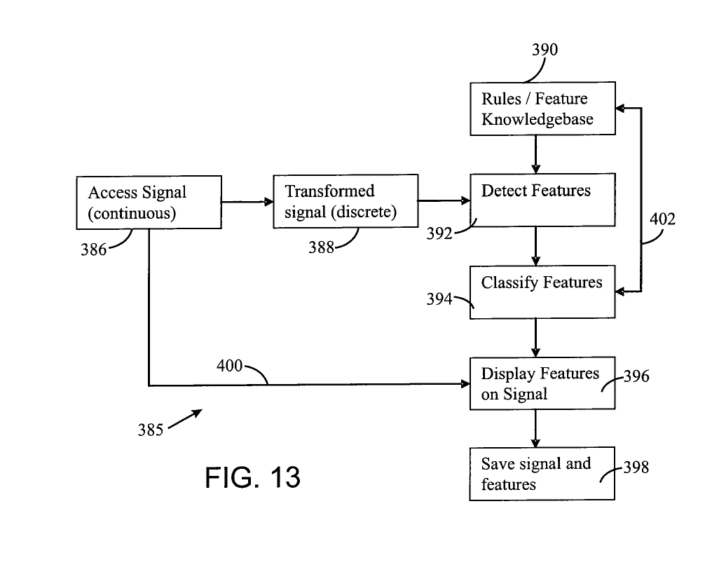

In a preferred embodiment, the signal detector comprises a signal transition detector shown in FIGS. 10 and 12 followed by an event classifier shown in FIG. 13. The signal transition detector 370 includes a comparator 382, which is presented with the signal V(t) and a time shifted copy of the signal V(t+Δt) 350, wherein the comparator identifies features in the signal that are distinguished by having a local zero derivative representing the change of direction of the signal amplitude. The output consists of digital pulses 354 of varying width as shown in FIG. 9.

The signal detector can be implemented using a circuit using conventional operational amplifiers for frequencies less than 200 Hz. However, for higher frequencies, comparator operational amplifiers are preferred. In any case, the output of the circuit is independent of the input signal. The method is sensitive to the time delay value 352, which will separate the signals in time. There are a number of conditions to consider in choosing the time delay value 352. It should prevent setting off events from small random noise amplitudes. It could be set to exclude certain portions of the cardiac signal time sequence. For example, when a good QRS signal is detected, a larger delay can be chosen.

In FIGS. 11 and 12, it can be seen that the waveforms and the amplitude transition threshold (deadband) 366 needed to trip the comparator 382 is a function of the associated hysteresis of the circuit, and the open loop gain of the comparator. The hysteresis amount ΔV is a function of the deadband 366 that can be chosen based on the component selection. The resistors R₁ 374 and R₂ 378 are chosen such that their ratio approximates the desired hysteresis. The components R378 and C380 determine the time constant of the delay. The threshold required to switch states is a function of the gain and slew rate of the comparator 382 or operational amplifier at the frequencies of interest. Typically the gain roll off rate is 20 dB per decade from 1 kHz onward. With such a roll off point, a 105 dB gain at 1 kHz reduces to a gain of 65 dB at 100 kHz. The slew rate is the maximum rate by which the output 384 can change state. For example, a IV/msec slew rate would require at least 5 ms to go from 0 to 5 volts, regardless how hard the input is being overdriven.

The output 384 of the detector is shown in FIG. 9 and it is a transformed signal which is discrete. It should be noted that this technique is immune to the variations in the input continuous signal unlike traditional methods. The discrete signal can be used advantageously for signal classification as described below.

In FIG. 13, signal classification is described. The signal classifier 385 has means 386 to access to the continuous analog biological signal which is transformed at block 388 into discrete signal by the signal detector described in FIGS. 9, 10, 11 and 12. The discrete signal is used to detecting the features of interest by a feature detection means 392. The feature detection means 392 compares the transformed signal to a previously determined rules/features knowledgebase. Based on previous determined features and the conditions at which a specific set of rules are applied, signals are put under different classes. Note that classifier 394 can also be linked to the knowledgebase 390 through a link 402 which may save results or expert overrides for the future references.

For the purposes of reporting on paper or in an electronic medical record, analog signal may be digitized and displayed or reported using the means 396 with the classified information superimposed via linkage 400. Finally the displayed signal can be stored and/or printed at block 398 for future reference.

In the case of fibrillation detection, the signal detector further comprises a pulse counter that counts the number of pulses for a preset time period. If the current signal corresponds to the normal heart beat, the pulse counter would register a count in a predetermined normal range since the normal biological signals have transition changes at a relatively low rate. In the event of a fibrillation, the count would be dramatically different and much higher than the normal rate and this increased count would be advantageously used to determine a defibrillation event. The physiological noise will also have relatively high counts but these counts would not add up to a sustained large number and thus can be differentiated from a fibrillation event. Unlike the traditional techniques, this method is robust and immune to signal filter degradations and provides a greatly improved event detection and classification.

As another example, the signal detector can be used to determine the heart rate and use this information in an algorithm for pacing a patient's heart. The heart rate detection is based on the number of transitions counted over a prespecified time interval. If the heart rate goes out of range for a predefined time and the frequency of the transitions remain in the non-fibrillation range, cardiac pacing can be initiated to pace the patient's heart.

In another application, when a discrete transition signal has been detected, it can be advantageously used to determine slope and slope duration analysis or any other methods of characterizing the QRS of an ECG signal.

Moreover, instead of the ECG, other signals may be used to utilize the disclosed concept. These may include blood pressure, vasomotor tone, electromyography (EMG), electrodermography, electroneuography, electro-oculography (EOG), electroretinography used (ERG), electronystagmography (ENG), video-oculography (VOG), infrared oculography (IROG), auditory evoked potentials (AEP), visual-evoked potentials (VEP), all kinds of Doppler signal, etc.

### Treatment:

The treatment system uses the information detected by the detection and classification system and treats the patient condition. The treatment can be imparted via electrical, mechanical, thermal, chemical or drug stimulation. In one embodiment, the treatment can be long term stimulation for tissue repair. In another embodiment the treatment is a periodic stimulation for chronic pain relief. In another example, the treatment is short term stimulation for defibrillating a fibrillating heart. In one embodiment, the treatment can be achieved by stimulating a vessel to treat a medical condition. In another embodiment, the treatment is achieved by stimulating a nerve indirectly through a vessel stimulation to treat a medical condition.

Certain changes to the existing means for the treatment are necessary before incorporating them in the intravascular framework. In the following, changes to the lead and the stimulation waveforms are described.

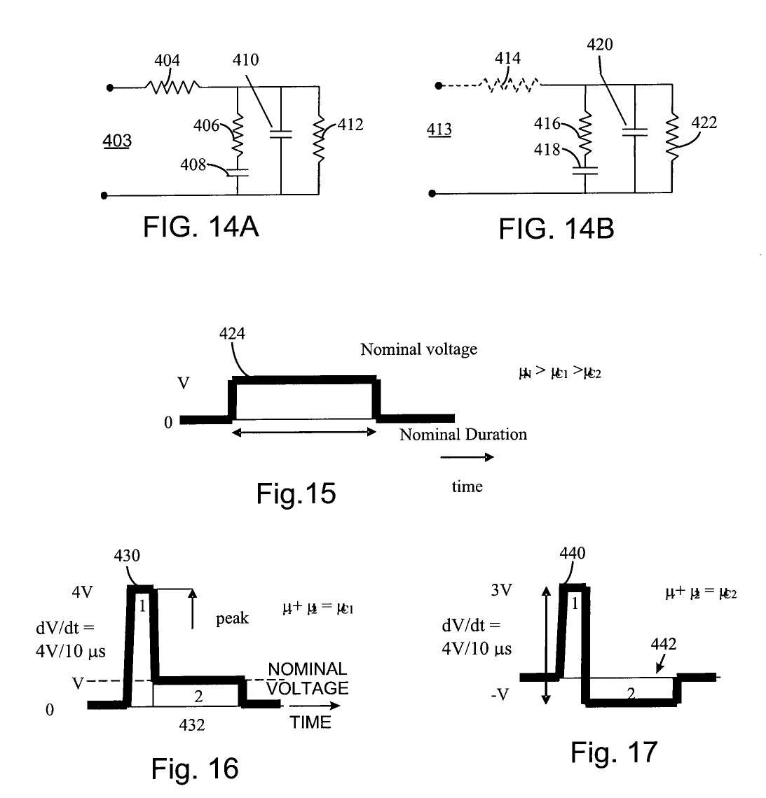

FIG. 14A is a schematic of circuitry of a traditional lead and the electrical equivalent of the tissue to be stimulated. The lead 403 is typically characterized by a high series resistance in the range of 200 to 1,500 ohms. The nominal value of this series resistance 404 is 1,200 ohms. The reason for this high resistance is to limit the current from a capacitor 410 (e.g., 7 μF). In order to represent the tissue resistance at DC, a resistance 412 is added in parallel to the capacitor 410. The electrical equivalent of the tissue to be stimulated is modeled as an equivalent resistance 406 and an equivalent capacitance 408 in parallel with the capacitance 410. The equivalent resistance is derived from a concatenated lattice comprising a series resistance and a capacitor connected to the commons.

FIG. 14B describes the present modification wherein the high resistance lead is replaced by an ultra low resistance lead 413 in a wireless intravascular platform. This is shown as a dotted component schematically. Preferably the resistance 414 of the lead is designed to be less than five Ohms. In order to represent the tissue resistance at DC, a resistance 422 is added in parallel to the capacitor 420. The current design makes the RC time constant smaller and consequently speeds up the rise time. This will be described next.

The stimulation waveform is generated using a computer program in the main computer of the intravascular platform. FIG. 15 describes a traditional pulse 424 that is characterized by a pulse of nominal amplitude that is “on” for a nominal duration (0.4 ms-2.0 ms). The area under the waveform is denoted by “N.”

FIG. 16 describes one embodiment of a composite pacing waveform diagram which is characterized by a first portion 430 consisting of a fast changing (4V/10 μs), short duration (0.05-0.2 ms), high amplitude (>3 times the nominal voltage) pulse that is followed by a second portion 432 consisting of a longer duration, pulse with an amplitude less than the nominal amplitude. The total duration of the pulse is less than the nominal duration of the traditional pulse. The total area under the first portion and the second portion is denoted by “C₁.” Note that area C₁ is less than the area N. Further note that the efficiency is gained by expending less overall energy and the clinical efficacy is gained by reducing the stimulation threshold for most of the duration of the pulse.

FIG. 17 describes another embodiment of a composite pacing waveform diagram which is characterized by a first portion 440 consisting of a fast changing (4V/10 μs), short duration (0.05-0.2 ms), high amplitude (>3 times the nominal voltage) pulse that is followed by a second portion 442 consisting of a longer duration, negative voltage pulse with an absolute amplitude that is less than the nominal amplitude. The total area under the first portion and the second portion is denoted by “C₂.” Again note that area C₂ is less than the area N. Further note that the efficiency is gained by expending even less overall energy and the clinical efficacy is gained by reducing the stimulation threshold for most of the duration of the pulse.

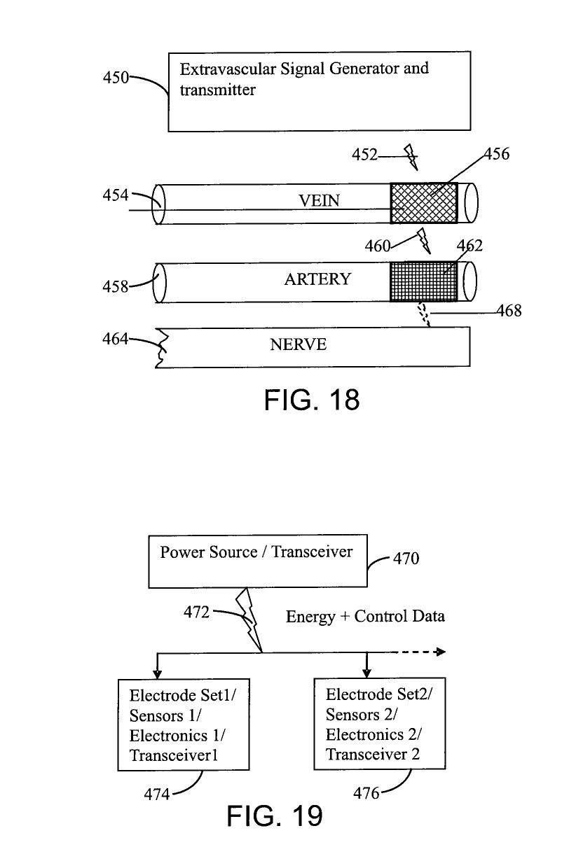

Getting back to alternative forms of stimulation, the stimulation treatment may be provided by stimulating a vessel indirectly through another vessel stimulation to treat a medical condition. FIG. 18 schematically illustrates functional aspects of this type of treatment system enabled by the intravascular platform.

In accordance with an exemplary method of this invention, one can utilize a system of RF energy transfer 452 from the extravascular signal generator and transmitter means 450 to the vascular transceiver 456 by use of wired or wireless means. Once the energy is received in the venous system it may be transmitted by means of more conventional wires 454 within the venous system. Such hardware is, in general, not a major issue in the venous aspect of the vasculature. However, such hardware can be problematic in the arterial aspect of the vasculature since it can potentially cause arterial occlusion or obstruction to arterial blood supply. RF energy is received into the venous vasculature as described before. The energy is then transferred to the arterial vessel 458 by means of inductive coupling 460 using, for example, parallel coils. The coil in the venous system is powered via the transceiver wired to a second site of interest. At this site, the artery is in close proximity contains a stent like coil 462 capable of receiving an induced current. This stent is not hardwired, but is placed similar to typical stents used to keep arteries open. However in this application the stent may have different configurations. In one embodiment, it may be an electrical solenoid type device. In another embodiment, it may be a spiral or a combination of spirals and solenoids. Any of these configurations are capable of converting the induced energy from the venous inductor for the purpose of stimulating receptors in the wall of the artery and/or for monitoring parameters such as pressure, blood flow and other physiologic parameters or chemical parameters in the artery. The arterial transceiver is also able to send such data either to the nearby transceiver in the venous system for relaying to the external receiver as shown or directly without a relay (not shown). In one embodiment, the stimulator coil 462 in the arterial system can stimulate a nerve 464 through energy transfer 468.

### Device Control:

The intravascular platform can be conveniently used to control a device. In one embodiment, the platform can deliver scalable wireless energy to one or more applications. In one example, the wireless energy can be used for powering a localized drug delivery system. As another example, the wireless energy can be used to control localized tissue ablation. In yet another example, the wireless energy may be used to control heart augmentation devices.

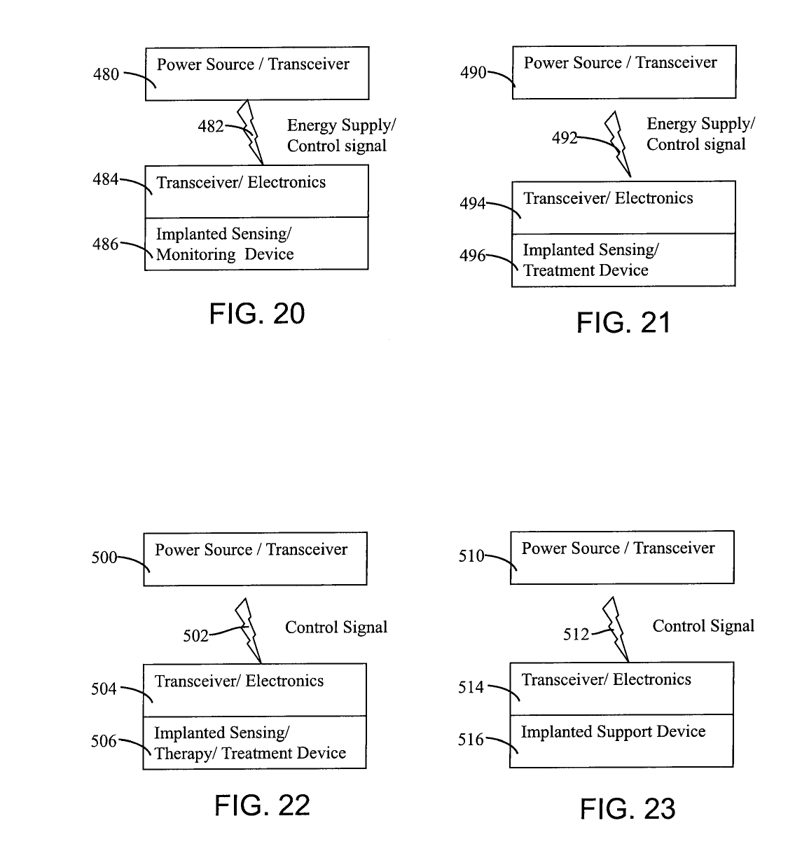

Referring to FIG. 20, the power source and the extravascular transceiver 480 can supply energy and control signals 482 through a transceiver/electronics system 484 to power an implanted sensing and/or monitoring system 486 that has been described in detail earlier.

Referring to FIG. 21, the power source and the extravascular transceiver 490 can supply energy and control signals 492 through a transceiver/electronics system 494 to power an implanted sensing and stimulation system 496 that has been described in detail earlier.

Referring to FIG. 22, the power source and the extravascular transceiver 500 can supply energy 502 through a transceiver/electronics system 504 to control an implanted sensing and stimulation system 506 that has been described in detail earlier.

Referring to FIG. 23, the power source and the extravascular transceiver 510 can supply energy 512 through a transceiver/electronics system 514 to control an implanted support system 516. For example, an implanted support system can be a cardiac augmentation device.

### Combination/Hybrid:

Multi-functional hybrid platform shown in the FIG. 19 can stimulate different sites using different site-specific electrodes and associated electronics. The power source and transceiver 470 can transfer energy and/or control data 472. The system further has integrated detection/sensing module such as 474 and 476 for one or more normal or abnormal medical conditions of one or more physiological processes and/or organ systems. One or more of multiple transceiver coils used for energy/data transfer and/or sensing/stimulation are programmatically selectable for the specific medical condition. The stimulation coil can be an energy relay coil to power a plurality of organ, tissue, fiber, molecular, and drug functions. The transmitter can send specific coded signals to select a specifically chosen receiver at a chosen site. In one embodiment, the hybrid system can perform multi-purpose cardiac stimulation that may include at least two treatments selected from a set comprising cardiac pacing and atrial fibrillation treatment and ventricular fibrillation treatment. In another embodiment, the hybrid system is applied to perform non-cardiac applications including brain stimulation, vagal nerve stimulation, spine stimulation, GERD treatment stimulation, GI tract stimulation, stimulation to treat obstructive airway disorders such as apnea, therapeutic stimulation of muscles, nervous tissue or organs, skeletal muscle stimulation, endotracheal stimulation, pelvic floor stimulation, sacral nerve stimulation, pancreatic stimulation, chronic constipation treatment, and prosthetic lamina stimulation for healing bone tissue. In another embodiment, the hybrid system can perform cardiac and non-cardiac stimulation.

With the hybrid platform it is possible to use any combination of features that are enabled by the system programmatically. It is also possible to disable any desired feature programmatically as well.

### Temporal Attributes of Wireless Intravascular Platform

In many applications, the intravascular wireless platform may be configured for various temporal attributes. In one example application, the wireless intravascular platform can be configured for continuous operation of, for example, monitoring or sensing. In another example, the platform may be configured for intermittent operation of, for example, electrical stimulation. This operation may be guided by a physiological need as in an exemplary case of cardiac stimulation of CHF patients whose heart rate fell below a predetermined threshold. In another example, the platform may be configured for a triggered operation. In this example, one may use intravascular or external ECG to trigger physiological data sensing and/or monitoring. In another example application, the platform may be operated by an interrupt. In this example, the platform may switch from the regular mode of operation to a time critical or life critical operation that requires immediate attention. Defibrillation therapy is an example of this case.

In some example applications, the platform may be interrogated to communicate with the external devices. The communication may be continuous, interval-based, interrupt driven, event driven or data driven. The communication may include by way of example, unprocessed or processed physiological data, alerts to the patient, or a caregiver, device service data, device identification data. The mode of communication may be audio, visual, text, or graphics. The communication may be local or remote. It may be automated, operator assisted or patient driven.

The foregoing description was primarily directed to a preferred embodiments of the invention. Although some attention was given to alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

We claim:

1. An intravascular system implantable in vascular of an animal for providing electrical stimulation of tissue to deal with a clinical condition of the animal, the intravascular system comprising:
an implantable control module for controlling the electrical stimulation and producing a desired digital waveform having an envelope defined as a function of an attribute;
an implantable intravascular sensing module sensing at least one parameter of interest for a purpose to deal with the clinical condition; and
an intravascular stimulation module for electrically stimulating the tissue with a output waveform that is substantially similar to the desired digital waveform produced by the implantable control module; and
a power supply for furnishing energy to the implantable control module, the implantable intravascular sensing module, and the intravascular stimulation module.

2. The intravascular system as recited in claim 1 wherein the power supply is an implantable non-rechargeable battery.

3. The intravascular system as recited in claim 1 wherein the power supply is an implantable rechargeable battery.

4. The intravascular system as recited in claim 1 wherein the power supply is a wireless energy source using a near-field resonant, inductive coupling to implantable components.

5. The intravascular system as recited in claim 1 wherein the at least one parameter of interest is related to a characteristic selected from a group consisting of electrical characteristics, mechanical characteristics, chemical characteristics, temperature, blood flow, blood pressure, blood volume, blood viscosity, electrolyte level, reference location, glucose level, urea level, carbon dioxide level, oxygen concentration, drug delivery, and drug level.

6. The intravascular system as recited in claim 1 wherein the purpose of electrical stimulation is selected from a group consisting of medical therapy, medical treatment, therapy monitoring, and detection or sensing of evoked responses to electrical stimulation of the tissue.

7. The intravascular system as recited in claim 1 wherein the clinical condition dealt with is selected from a group consisting of irregular cardiac rhythms, slow or fast cardiac rhythms, infarct repair, ischemia detection, chronic heart failure resynchronization, tachycardia stimulation/cardiac stimulation, seizure prevention, seizure warning, obsessive compulsive disorder, spine problem, GERD, neuronal disorder, gastro-intestinal disorder, obstructive airway disorder, skeletal muscle problem, endo tracheal problem, pelvic floor problem, sacral nerve problem, depression, obesity, pain relief, nerve damage, pancreatic disorder, chronic constipation problem, and internal wounds.

8. The intravascular system as recited in claim 1 wherein the intravascular stimulation module is adapted to electrically stimulate tissue of an animal organ selected from a group consisting of brain, heart, esophagus, stomach, kidney, ear, eye, lung, uterus, prostate, blood, spine, bladder, pancreas, colon, and nervous system.

9. The intravascular system as recited in claim 1 wherein the output waveform is at least one of intermittent, interrupt driven, and event driven.

10. The intravascular system as recited in claim 1 wherein the attribute is one of a physiological characteristic of the animal, received telemetry signal, and a preprogrammed clinical algorithm.

11. A wireless intravascular system implantable in vascular of an animal for providing electrical stimulation of tissue to deal with a clinical condition of the animal, the intravascular system comprising:
an implantable control module for controlling operation of the wireless intravascular system and producing a desired digital waveform having an envelope that is a function of an attribute;
an implantable intravascular sensing module sensing at least one parameter of interest for a purpose to deal with the clinical condition; and
an intravascular stimulation module for electrically stimulating the tissue with a output waveform that is substantially similar to the desired digital waveform produced by the implantable control module; and
a power supply module utilizing a near-field resonant, inductive coupling to convey energy to the implantable control module, the implantable intravascular sensing module, and the intravascular stimulation module.

12. The wireless intravascular system as recited in claim 11 wherein the at least one parameter of interest is related to a characteristic selected from a group consisting of temperature, blood pressure, blood volume, blood flow, blood viscosity, electrical characteristics, mechanical characteristics, chemical characteristics, electrolyte level, reference location, glucose level, urea level, carbon dioxide level, oxygen concentration, carbon dioxide level, drug delivery, and drug level.

13. The wireless intravascular system as recited in claim 11 wherein the purpose of electrical stimulation is selected from a group consisting of medical therapy, medical treatment, therapy monitoring, and detection or sensing of evoked responses to electrical stimulation of the tissue.

14. The wireless intravascular system as recited in claim 11 wherein the clinical condition dealt with is selected from a group consisting of irregular cardiac rhythms, slow or fast cardiac rhythms, infarct repair, ischemia detection, tachycardia stimulation/cardiac stimulation, chronic heart failure resynchronization, seizure prevention, seizure warning, obsessive compulsive disorder, spine problem, GERD, neuronal disorder, gastrointestinal disorder, obstructive airway disorder, skeletal muscle problem, endo tracheal problem, pelvic floor problem, sacral nerve problem, depression, obesity, pain relief, nerve damage, pancreatic disorder, chronic constipation problem, and internal wounds.

15. The wireless intravascular system as recited in claim 11 wherein the intravascular stimulation module is adapted to electrically stimulate tissue of an animal organ selected from a group consisting of brain, heart, esophagus, stomach, kidney, ear, eye, lung, uterus, prostate, blood, spine, bladder, pancreas, colon, and nervous system.

16. The wireless intravascular system as recited in claim 11 wherein the output waveform is at least one of intermittent, interrupt driven, and event driven.

17. The intravascular system as recited in claim 11 wherein the attribute is one of a physiological characteristic of the animal, received telemetry signal, and a preprogrammed clinical algorithm.

18. An intravascular implantable system for providing electrical stimulation of a tissue in side an animal for to deal with a clinical condition, the intravascular implantable system comprising:
an implantable control module for controlling the electrical stimulation and producing desired digital waveforms that have envelopes with predetermined attributes;
an implantable intravascular sensing module sensing at least one parameter of interest for a purpose of dealing with the clinical condition; and
an intravascular stimulation module electrically stimulating the tissue with a output waveform that is substantially similar to the desired digital waveform produced by the implantable control module; and
a power supply module comprising a rechargeable energy source with a near-field resonant, inductive coupling for supplying energy to the implantable control module, the implantable intravascular sensing module, and the intravascular stimulation module.

19. The intravascular implantable system as recited in claim 18 wherein the at least one parameter of interest is related to a characteristic selected from a group consisting of temperature, blood pressure, blood volume, blood flow, blood viscosity, electrical characteristics, mechanical characteristics, chemical characteristics, electrolyte level, reference location, glucose level, urea level, carbon dioxide level, oxygen concentration, carbon dioxide level, drug delivery, and drug level.

20. The intravascular implantable system as recited in claim 18 wherein the purpose of electrical stimulation is selected from a group consisting of medical therapy, medical treatment, therapy monitoring, and detection or sensing of evoked responses to electrical stimulation of the tissue.

21. The intravascular implantable system as recited in claim 18 wherein the clinical condition dealt with is selected from a group consisting of irregular cardiac rhythms, slow or fast cardiac rhythms, infarct repair, ischemia detection, tachycardia stimulation/cardiac stimulation, chronic heart failure resynchronization, seizure prevention, seizure warning, obsessive compulsive disorder, spine problem, GERD, neuronal disorder, gastrointestinal disorder, obstructive airway disorder, skeletal muscle problem, endotracheal problem, pelvic floor problem, sacral nerve problem, depression, obesity, pain relief, nerve damage, pancreatic disorder, chronic constipation problem, and internal wounds.

22. The intravascular implantable system as recited in claim 18 wherein the intravascular stimulation module is adapted to electrically stimulate tissue of an animal organ selected from a group consisting of brain, heart, esophagus, stomach, kidney, ear, eye, lung, uterus, prostate, blood, spine, bladder, pancreas, colon, and nervous system.

23. The intravascular implantable system as recited in claim 18 wherein the output waveform is at least one of intermittent, interrupt driven, and event driven.

24. The intravascular system as recited in claim 18 wherein the attribute is one of a physiological characteristic of the animal, received telemetry signal, and a preprogrammed clinical algorithm.
